(19) United States
(12) Patent Application Publication
(10) Pub. No.: US 2025/0354098 A1
(43) Pub. Date: Nov. 20, 2025

(54) DIGITAL MICROFLUIDIC DEVICES AND ASSOCIATED METHODS

(71) Applicants: Sartorius Stedim Cellca GmbH, Ulm (DE); The Governing Council of the University of Toronto, Toronto (CA)

(72) Inventors: Samin Akbari, Winchester, MA (US); David James Pollard, Stow, MA (US); Richard Wales, St. Neots (GB); Ian Swyer, South Pickering (CA); Michael Dean Chamberlain, Toronto (CA); Sheldon Decombe, Toronto (CA); Aaron Wheeler, Toronto (CA); Jeremy Thomas Lant, Toronto (CA); Mahmoud Ahmed Abdelalim, Toronto (CA); Jurgen Frasheri, Toronto (CA)

(73) Assignees: Sartorius Stedim Cellca GmbH, Ulm (DE); The Governing Council of the University of Toronto, Toronto (CA)

(21) Appl. No.: 18/868,704

(22) PCT Filed: May 17, 2023
(86) PCT No.: PCT/EP2023/063310

§ 371 Date: Nov. 22, 2024

Related U.S. Application Data
(60) Provisional application No. 63/345,375, filed on May 24, 2022.

(51) Int. Cl. C12M 3/06; C12M 1/00; C12M 1/26; C12M 1/34

(52) U.S. Cl. CPC C12M 23/16; C12M 23/40; C12M 33/14; C12M 41/00

(57) ABSTRACT

The present disclosure generally relates to digital microfluidic devices and associated methods. Some digital microfluidic devices described herein may be particularly suitable for manipulating droplets suitable for hosting cell growth. For instance, some digital microfluidic devices may include one or more features that assists with measuring and/or adjusting a property of one or more droplets during cell growth. As another example, some digital microfluidic devices may include one or more features that assist with performing a time-series measurement of one or more properties of a population of cells growing in a droplet. Such digital microfluidic devices may advantageously allow cell growth conditions to be recorded and/or adjusted during cell growth, which may enhance understanding of how various parameters affect cell growth and/or control of cell growth based on such knowledge.

### RELATED APPLICATIONS

This application claims priority under 35 U.S.C. § 119 (e) to U.S. Provisional Application No. 63/345,375, filed May 24, 2022, and entitled “Digital Microfluidic Devices,” which is incorporated herein by reference in its entirety for all purposes.

### FIELD

Digital microfluidic devices and associated methods are generally provided.

### BACKGROUND

In many cases, it can be desirable to grow cells in one or more droplets. During such processes, it may also be beneficial to measure and/or adjust one or more droplet properties during cell growth. However, current systems for growing cells in droplets have drawbacks. Accordingly, new digital microfluidic devices and associated methods are needed.

### SUMMARY

The present disclosure generally describes digital microfluidic devices. The subject matter described herein involves, in some cases, interrelated products, alternative solutions to a particular problem, and/or a plurality of different uses of one or more systems and/or articles.

In some embodiments, a digital microfluidic device is provided. The digital microfluidic device comprises a base substrate, a top substrate spaced from the base substrate, a plurality of electrodes positioned on a side of the base substrate facing the top substrate, a sensor positioned on a side of the base substrate facing the top substrate, an electrode positioned on a side of the top substrate facing the base substrate, a first coating disposed on the plurality of electrodes positioned on the base substrate, and a second coating disposed on the electrode positioned on the top substrate. The electrodes positioned on the base substrate and/or the top substrate are configured to translate a droplet positioned between the base substrate and the top substrate across a plurality of locations. The plurality of locations comprises a location associated with the sensor. The sensor is configured to sense a property of a droplet and to communicate the sensed property to a controller/control system. The digital microfluidic device is in fluidic communication with a reagent source. The controller is configured to send the digital microfluidic device instructions to adjust one or more properties of the droplet based on the property sensed by the sensor.

In some embodiments, a digital microfluidic device comprises a base substrate, a top substrate spaced from the base substrate, a plurality of electrodes positioned on a side of the base substrate facing the top substrate, a sensor positioned on a side of the base substrate facing the top substrate, an electrode positioned on a side of the top substrate facing the base substrate, a first coating disposed on the plurality of electrodes positioned on the base substrate, and a second coating disposed on the electrode positioned on the top substrate. The electrodes positioned on the base substrate and/or the top substrate are configured to translate a droplet positioned between the base substrate and the top substrate across a plurality of locations. The plurality of locations comprises a location associated with the sensor. The sensor is configured to determine the number of cells present in the droplet and/or the viability of cells present in the droplet. The digital microfluidic device is configured to perform the measurements at a frequency of at least once per hour. The digital microfluidic device is configured to perform the measurements over a total period of time of at least 1 day.

In some embodiments, a method is provided. The method is performed in a digital microfluidic device comprising a base substrate, a top substrate spaced from the base substrate, a plurality of electrodes positioned on a side of the base substrate facing the top substrate, an electrode positioned on a side of the top substrate facing the base substrate, a first coating disposed on the plurality of electrodes positioned on the base substrate, a second coating disposed on the electrode positioned on the top substrate, and a sensor positioned on a side of the base substrate facing the top substrate. The method comprises employing the electrodes positioned on the base substrate and/or the top substrate to translate a droplet positioned between the base substrate and the top substrate across a plurality of locations, wherein the plurality of locations comprises a location associated with the sensor, sensing a property of the droplet with the sensor, communicating the sensed property of the droplet to a controller, employing the controller to send the digital microfluidic device instructions to adjust one or more properties of the droplet based on the property sensed by the sensor, and adjusting one or more properties of the droplet based on the property sensed by the sensor. The one or more properties of the droplet are adjusted by supplying the droplet with a reagent from the reagent source.

In some embodiments, a method is performed in a digital microfluidic device comprising a base substrate, a top substrate spaced from the base substrate, a plurality of electrodes positioned on a side of the base substrate facing the top substrate, an electrode positioned on a side of the top substrate facing the base substrate, a first coating disposed on the plurality of electrodes positioned on the base substrate, a second coating disposed on the electrode positioned on the top substrate, and a sensor positioned on a side of the base substrate facing the top substrate. The method comprises employing the electrodes positioned on the base substrate and/or the top substrate to translate a droplet positioned between the base substrate and the top substrate across a plurality of locations, wherein the plurality of locations comprises a location associated with a sensor configured to determine the number of cells present in the droplet and/or the viability of cells present in the droplet and employing the sensor to perform a plurality of measurements of the number of cells present in the droplet and/or the viability of the cells present in the droplet. The measurements are performed at a frequency of at least once per hour. The plurality of measurements is performed over a total period of time of at least 1 day.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 depicts a digital microfluidic device, in accordance with some embodiments;

FIG. 2 depicts a digital microfluidic device in which a droplet is positioned, in accordance with some embodiments;

FIG. 3 depicts a digital microfluidic device comprising electrodes, in accordance with some embodiments;

FIG. 4 depicts a digital microfluidic device comprising an electrode positioned on the top substrate, in accordance with some embodiments;

FIG. 5 depicts a digital microfluidic device comprising coatings disposed on the base and top substrates, in accordance with some embodiments;

FIG. 6 depicts a digital microfluidic device comprising a dielectric, in accordance with some embodiments;

FIG. 7 depicts a digital microfluidic device comprising a sensor, in accordance with some embodiments;

FIG. 8 depicts a digital microfluidic device comprising a reagent source, in accordance with some embodiments;

FIG. 9 depicts a digital microfluidic device in electrical communication with a controller, in accordance with some embodiments;

FIG. 10 depicts a digital microfluidic device that comprises an optical detector, in accordance with some embodiments;

FIG. 11 depicts a top view of a digital microfluidic device that comprises a filtration assembly, in accordance with some embodiments;

FIG. 12 depicts images of cells filtered on a digital microfluidic device with a paper-based filter, in accordance with some embodiments;

FIG. 13 depicts images of cells filtered on a digital microfluidic device with a hydrogel-based filter, in accordance with some embodiments;

FIG. 14 depicts images of cells filtered on a digital microfluidic device with a porous polymer-based filter and corresponding data, in accordance with some embodiments;

FIG. 15 depicts a side view of a digital microfluidic device that comprises a filtration assembly and a plurality of electrodes, in accordance with some embodiments;

FIG. 16 depicts a view of a digital microfluidic device that comprises a filtration assembly, a plurality of electrodes, and a vessel, in accordance with some embodiments;

FIG. 17 depicts images of an example of filtration occurring through holes on a digital microfluidic substrate, in accordance with some embodiments;

FIG. 18 depicts an exploded view of an enclosure for a digital microfluidic device, in accordance with some embodiments;

FIG. 19 depicts a top view of a bulk reagent dispensing system, in accordance with some embodiments;

FIG. 20 depicts a side view of the bulk reagent dispensing manifold illustrated in FIG. 19, in accordance with some embodiments;

FIG. 21 depicts images of an embodiment of a digital microfluidic device enclosure with a humidity chamber, and data associated therewith, in accordance with some embodiments;

FIG. 22 depicts a top view of a waste removal microfluidic manifold, in accordance with some embodiments;

FIG. 23 depicts a side view of the waste removal microfluidic manifold illustrated in FIG. 22, in accordance with some embodiments; and

FIG. 24 depicts images of examples of patterned microstructure-based filtration assemblies fabricated for use on digital microfluidic devices, in accordance with some embodiments.

### DETAILED DESCRIPTION

The present disclosure generally relates to digital microfluidic devices and associated methods. Some digital microfluidic devices described herein may be particularly suitable for manipulating droplets suitable for hosting cell growth. For instance, some digital microfluidic devices may include one or more features that assists with measuring and/or adjusting a property of one or more droplets during cell growth. As another example, some digital microfluidic devices may include one or more features that assist with performing a time-series measurement of one or more properties of a population of cells growing in a droplet. Such digital microfluidic devices may advantageously allow cell growth conditions to be recorded and/or adjusted during cell growth, which may enhance understanding of how various parameters affect cell growth and/or control of cell growth based on such knowledge. Such digital microfluidic devices may also allow for the growth of multiple different types of cells in different droplets, which may advantageously allow for rapid screening of how different types of cells respond to the same cell culture conditions.

Digital microfluidic devices may comprise a plurality of components that are suitable for generating droplets, manipulating droplets, monitoring droplets, and/or allowing cells to grow in droplets. In some embodiments, a digital microfluidic device is capable of receiving digital instructions and/or sending digital information. It is also possible for the digital microfluidic devices described herein to be capable of manipulating droplets in a digital manner. For instance, in some embodiments, the digital microfluidic devices herein may be configured to locate droplets in a plurality of discrete positions, may be capable of locating droplets in a plurality of discrete positions, and/or may locate droplets in a plurality of discrete positions. The digital microfluidic devices described herein may be configured to manipulate, may be capable of manipulating, and/or may manipulate microfluidic quantities of fluid (e.g., quantities of fluid having volumes of 1 mL or less).

Various features of some digital microfluidic devices are described below with respect to the figures. It should be noted that although some FIGS. show relatively few features of digital microfluidic devices and some FIGS. show a relatively large number of such features, the digital microfluidic devices described herein should be understood to possibly comprise some, all, or none of the features shown in any particular FIG. Additionally, some digital microfluidic devices may comprise a combination of features shown in two or more FIGS. but not shown together in a single FIG.

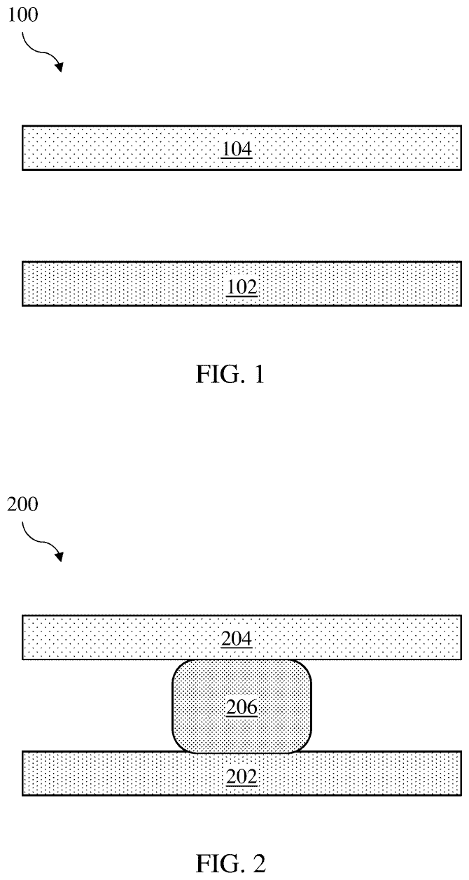

FIG. 1 shows one non-limiting embodiment of a digital microfluidic device 100. This digital microfluidic device comprises a base substrate 102 and a top substrate 104. It should be understood that, although FIG. 1 depicts the base substrate as being positioned beneath the top substrate, other arrangements of the base substrate with respect to the top substrate are also possible. As an example, in some embodiments, a digital microfluidic device is arranged so that a base substrate is positioned above the top substrate. It is also possible for a digital microfluidic device to be arranged so that the base substrate is positioned beside the top substrate. In such embodiments, features that are described herein as being on a side of the base substrate facing the top substrate (or an upper side of the base substrate) may still be arranged such that they are on a side of the base substrate facing the top substrate (even if that side is not an “upper side” of the base substrate in the orientation in which the digital microfluidic device is positioned). Similarly, in such embodiments, features that are described herein as being on a side of the top substrate facing the base substrate (or a lower side of the top substrate) may still be arranged such that they are on a side of the top substrate facing the base substrate (even if that side is not a “lower side” of the top substrate in the orientation in which the digital microfluidic device is positioned). For instance, a digital microfluidic device described herein may be positioned and/or operated in a manner that is “upside down” or “rotated” from the orientations shown in the FIGS., whereby a base substrate comprising of a plurality of electrodes facing a top substrate is positioned on top.

In some embodiments, digital microfluidic devices include transparent windows to facilitate imaging and transmission of light on both sides of the enclosure for imaging at sites of interest. In this embodiment, there is bare glass (instead of patterned electrodes) on base substrates of digital microfluidic devices allowing imaging at that location.

The digital microfluidic devices described herein may be employed to perform one or more operations on droplets and/or to generate droplets. In some embodiments, a base substrate and a top substrate of a digital microfluidic device are spaced so that a droplet may be positioned between them (e.g., the droplet 206 in FIG. 2). Such droplets may extend across the full thickness of the space between the base and top substrates (e.g., like the droplet shown in FIG. 2), or may have a height such that they are not in contact with the top substrate (not shown). The fluid surrounding the droplet (e.g., enclosed between the base substrate 202 and the top substrate 204 and surrounding the droplet 206) may be selected as desired. In some embodiments, the fluid may be air. It is also possible for the fluid to be an oil.

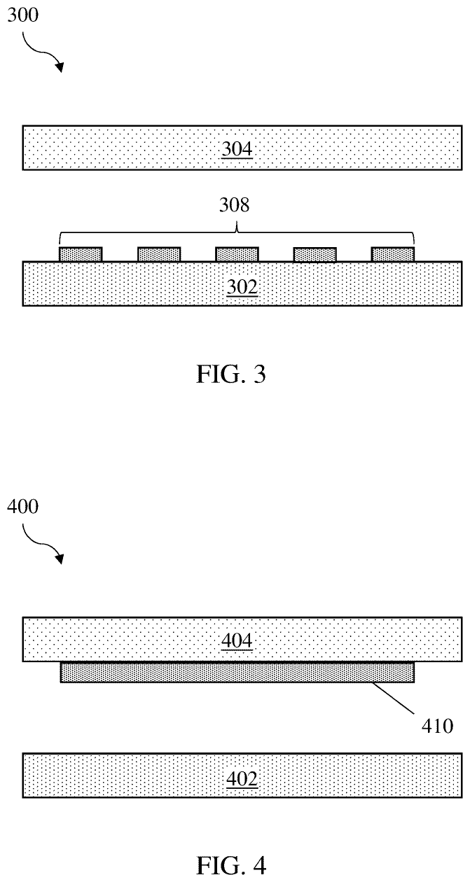

Droplet generation and/or droplet manipulation may be performed with the assistance of an electric field. The electric field may be created by generating a potential difference between two or more electrodes located on the digital microfluidic device. The electrodes between which the potential difference is generated may be electrically insulated from each other. Accordingly, some digital microfluidic devices may comprise one or more electrodes (e.g., two or more electrodes that are electrically insulated from each other). FIG. 3 shows one non-limiting example of such a digital microfluidic device. In FIG. 3, the digital microfluidic device 300 comprises a plurality of electrodes 308. The electrodes in FIG. 3 are positioned on the base substrate on a side thereof that faces the top substrate. It is also possible for a digital microfluidic device to comprise an electrode positioned on the top substrate (e.g., on a side of the top substrate facing the base substrate). FIG. 4 depicts one such embodiment. In some embodiments, a digital microfluidic device comprises both an electrode positioned on the top substrate and a plurality of electrodes positioned on the base substrate. In such embodiments, a potential difference may be generated between the electrode positioned on the top electrode and one or more of the electrodes positioned on the base substrate.

In some embodiments, a plurality of electrodes positioned on a base substrate are a plurality of driving electrodes and an electrode positioned on a top substrate is a ground electrode. However, other configurations of electrodes are also possible, such as configurations in which one or more driving electrodes are positioned on a top substrate and/or a ground electrode is positioned on a base substrate.

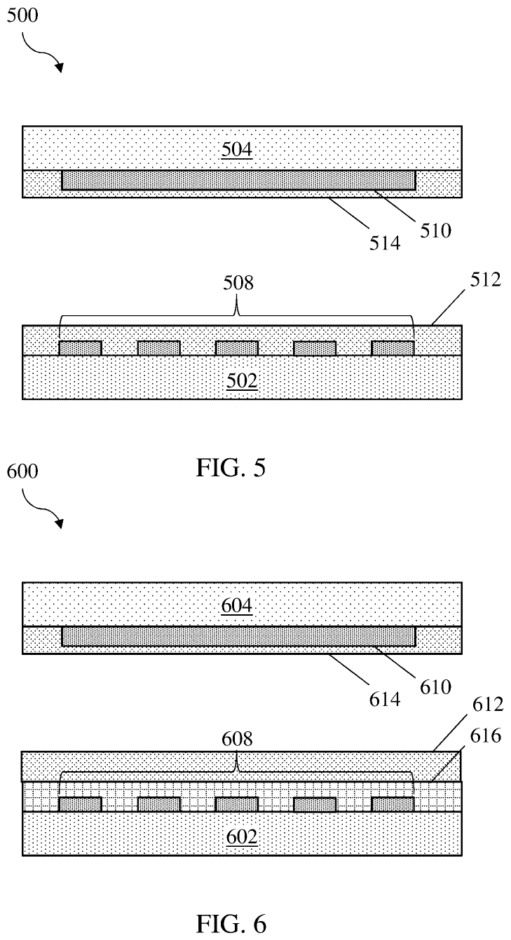

In some embodiments, a digital microfluidic device comprises one or more coatings disposed on a surface of a base substrate that faces a top substrate and/or on a surface of a top substrate that faces a base substrate. Coatings may assist with droplet generation, droplet manipulation, droplet stability, and/or cell growth in a droplet. Some coatings may be the outermost layer on the top substrate or the base substrate. In such embodiments, any droplets positioned between the base substrate and the top substrate may directly contact the coating(s) disposed thereon if positioned in a portion of the digital microfluidic device comprising the coating. FIG. 5 shows one non-limiting example of a digital microfluidic device comprising the coatings 512 and 514 disposed on the base and top substrates, respectively.

Some digital microfluidic devices may comprise a dielectric positioned between a coating and a base substrate or a top substrate. When present, the dielectric may assist with electrically insulating electrodes disposed on a base substrate and/or a top substrate from each other. This may be advantageous when a droplet positioned between an electrode on a base substrate and an electrode positioned on a top substrate would otherwise contact these electrodes and cause a short circuit. FIG. 6 shows one example of a digital microfluidic device comprising a dielectric. In FIG. 6, the digital microfluidic device 600 comprises a dielectric 616 positioned between the coating 612 and the base substrate 602. In FIG. 6, the coating is positioned between the plurality of electrodes positioned on the base substrate and the coating disposed on the base substrate. It is also possible, additionally or alternatively, for a digital microfluidic device to comprise a dielectric positioned between a top substrate and a coating thereon (e.g., positioned between an electrode and a coating disposed on a top substrate).

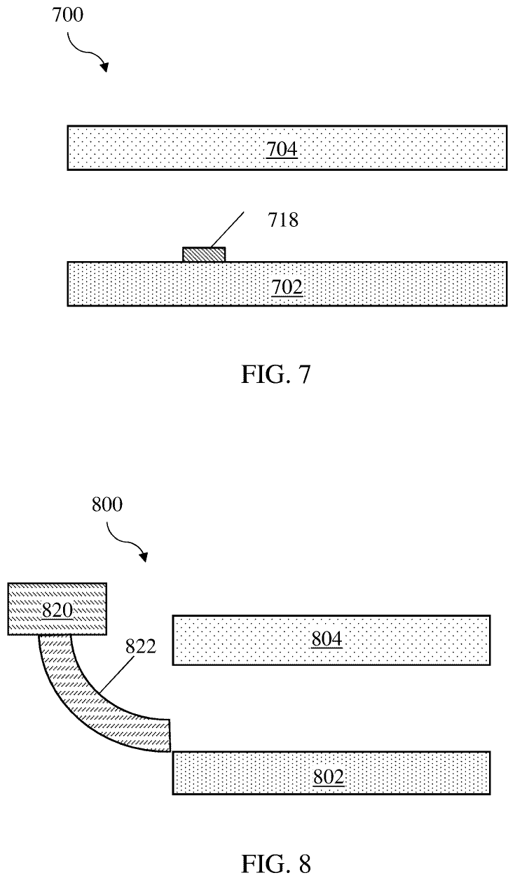

In some embodiments, a digital microfluidic device comprises one or more sensors. Such sensors may be configured to sense, be capable of sensing, and/or sense one or more properties of the droplet(s) present in the digital microfluidic device. Advantageously, such sensors may be capable of being employed, be configured to be employed, and/or be employed to detect the relevant property or properties over time (e.g., to measure the change in one or more droplet properties over time). It is also possible for such sensors to be capable of being employed, configured to be employed, and/or be employed as part of an active feedback loop (e.g., with the assistance of a controller) that maintains the property or properties at a relatively constant value and/or within a range. FIG. 7 shows one example of a digital microfluidic device 700 comprising a sensor 718. It is also possible for a digital microfluidic device to comprise two or more sensors positioned at different locations (not shown). In embodiments in which a digital microfluidic device comprises both a coating and a sensor positioned on a substrate (e.g., a base substrate), the coating may be positioned between the sensor and the substrate and/or the sensor may be positioned between the coating and the substrate.

As described above, some digital microfluidic devices described herein include two or more sensors. In such embodiments, each sensor may be configured to, may be capable of, and/or may sense different properties. It is also possible for each sensor to be configured to, be capable of, and/or sense the same property. In some embodiments, a digital microfluidic device comprises two or more sensors that are configured to, are capable of, and/or sense different properties from each other and also comprise two or more sensors that are configured to, are capable of, and/or sense the same property (or properties) as each other.

In some embodiments, a sensor is configured to determine, is capable of determining, and/or determines one or more features of any cells present in a droplet. As one example, a sensor may be configured to determine, may be capable of determining, and/or may determine the number of cells present in a droplet (e.g., a droplet with which it is associated). As another example, a sensor may be configured to determine, may be capable of determining, and/or may determine the viability of cells present in a droplet. Such sensors may be optical sensors. As an example, a sensor may comprise an optical fiber, a light source, a microscope, and/or a camera. Such sensors may also comprise one or more computer programs that are capable of determining, configured to determine, and/or determine the feature(s) of the cells. Further non-limiting examples of suitable sensors include pH sensors, dissolved oxygen sensors, carbon dioxide sensors, glucose sensors, protein titer sensors (e.g., ELISA), and metabolite sensors (e.g., lactate sensors, glutamine sensors, glutamate sensors, ammonium sensors, potassium sensors). In some embodiments, an optical sensor may be employed to determine one or more of the above-described properties of a droplet. As one example, an optical sensor may be employed to determine the pH of a droplet comprising a colorimetric and/or fluorescent pH indicator. As another example, an optical sensor may be employed to determine the results of an ELISA assay that yields an optically-detectable result.

It is also possible for a digital microfluidic device to comprise a reagent source. In some embodiments, a digital microfluidic device does not comprise a reagent source but is configured to be, is capable of being, and/or is in fluidic communication with a reagent source. The fluidic communication may be reversible or irreversible. The reagent source may be configured to provide, capable of providing, and/or provide one or more reagents to one or more droplets present in the digital microfluidic device. For instance, the reagent source may be configured to be, capable of being, and/or be in fluidic communication with one or more portions of the digital microfluidic device in which one or more droplets are present. Reagents provided by a reagent source may be configured to be, capable of being, and/or be employed to assist with maintaining one or more droplet properties at a relatively constant value or within a range or to change one or more droplet properties (e.g., droplet pH, media concentration in the droplet, presence and/or concentration of one or more reagents in the droplet, presence of one or more reagents and/or buffers for performing an assay, such as an ELISA assay). FIG. 8 depicts one example of a digital microfluidic device comprising a reagent source. In FIG. 8, the digital microfluidic device 800 comprises a reagent source 820. The reagent source is in fluidic communication with the portion of the device between the base substrate 802 and the top substrate 804 via a fluidic conduit 822.

Some digital microfluidic devices may comprise, be configured to be in fluidic communication with, be capable of being in fluidic communication with, and/or be in fluidic communication with a reservoir that is not a reagent source (not shown). Reservoirs may contain fluids suitable for use in the digital microfluidic device other than reagents supplied by a reagent source, such as solutions suitable for cleaning the interior of the digital microfluidic device (and/or one or more surfaces therein).

In some embodiments, a digital microfluidic device comprises a controller, is configured to be in communication (e.g., electrical communication, electromagnetic communication), is capable of being in communication, and/or is in communication with a controller.

In such embodiments, the controller may be configured to send, may be capable of sending, and/or may send the digital microfluidic device instructions. For instance, a controller may be configured to send, may be capable of sending, and/or may send instructions to adjust one or more properties of the droplet and/or of the digital microfluidic device. As another example, a controller may be configured to send, may be capable of sending, and/or may send instructions to translate one or more droplets in the digital microfluidic device (e.g., by generating and/or adjusting an electric field present inside the digital microfluidic device) and/or to stop translating one or more droplets in the digital microfluidic device (e.g., by terminating and/or adjusting an electric field present inside the digital microfluidic device).

It is also possible for a controller to be configured to receive, be capable of receiving, and/or receive information from the digital microfluidic device. As an example, in some embodiments, a controller is configured to receive, is capable of receiving, and/or receives information from one or more sensors present in the digital microfluidic device. Upon receiving information from a sensor, the controller may communicate that information (e.g., to user, to another device), process that information, store that information, and/or send instructions to the digital microfluidic device based on that information. It is also possible for the controller to be capable of and/or configured to do some or all of the foregoing. For instance, a controller may be configured to, may be capable of, and/or may do one or more of the following: perform calculations on information received from the sensor, store information received from the sensor and/or the results of calculations performed on information received from the sensor, and/or display information received from the sensor.

A controller may comprise a computer implemented control system. The computer implemented control system may be configured to perform, may be capable of performing, and/or may perform one or more of the actions described in the preceding paragraphs.

A computer implemented control system may include several known components and circuitry, including a processing unit (e.g., a processor), a memory system, input and output devices and interfaces (e.g., an interconnection mechanism), transport circuitry (e.g., one or more busses), a video and/or audio data input/output (I/O) subsystem, and/or special-purpose hardware. Further, the computer implemented control system may be a multi-processor computer system or may include multiple computers connected over a computer network.

When present, a computer implemented control system may include a processor, for example, a commercially available processor such as one of the series x86, Celeron and Pentium processors, available from Intel, similar devices from AMD and Cyrix, the 680X0 series microprocessors available from Motorola, and the PowerPC microprocessor from IBM. Many other processors are available, and the computer system is not limited to a particular processor.

A processor typically executes a program called an operating system, of which WindowsNT, Windows 95 or 98, UNIX, Linux, DOS, VMS, MacOS and OS8 are examples, which controls the execution of other computer programs and provides scheduling, debugging, input/output control, accounting, compilation, storage assignment, data management and memory management, communication control and/or related services. The processor and operating system together may define a computer platform for which application programs in high-level programming languages are written. The computer implemented control system is not limited to a particular computer platform.

The computer implemented control system may include a memory system, which typically includes a computer readable and writeable non-volatile recording medium, of which a magnetic disk, optical disk, a flash memory and tape are examples. Such a recording medium may be removable, for example, a floppy disk, read/write CD or memory stick, or may be permanent, for example, a hard drive.

Such a recording medium stores signals, typically in binary form (i.e., a form interpreted as a sequence of one and zeros). A disk (e.g., magnetic or optical) has a number of tracks, on which such signals may be stored, typically in binary form. Such signals may define a software program, e.g., an application program, to be executed by the microprocessor, or information to be processed by the application program.

The memory system of a computer implemented control system also may include an integrated circuit memory element, which typically is a volatile, random access memory such as a dynamic random access memory (DRAM) or static memory (SRAM). Typically, in operation, the processor causes programs and data to be read from the non-volatile recording medium into the integrated circuit memory element, which typically allows for faster access to the program instructions and data by the processor than does the non-volatile recording medium.

The processor generally manipulates the data within the integrated circuit memory element in accordance with the program instructions and then copies the manipulated data to the non-volatile recording medium after processing is completed. A variety of mechanisms are known for managing data movement between the non-volatile recording medium and the integrated circuit memory element, and the computer implemented control system that implements the methods described above is not limited thereto. The computer implemented control system is not limited to a particular memory system.

At least part of such a memory system described above may be used to store one or more data structures (e.g., look-up tables) and/or equations. For example, at least part of a non-volatile recording medium may store at least part of a database that includes one or more of such data structures. Such a database may be any of a variety of types of databases, for example, a file system including one or more flat-file data structures where data is organized into data units separated by delimiters, a relational database where data is organized into data units stored in tables, an object-oriented database where data is organized into data units stored as objects, another type of database, and/or any combination thereof.

A computer implemented control system may include a video and audio data I/O subsystem. An audio portion of the subsystem may include an analog-to-digital (A/D) converter, which receives analog audio information and converts it to digital information. The digital information may be compressed using known compression systems for storage on the hard disk to use at another time. A typical video portion of the I/O subsystem may include a video image compressor/decompressor of which many are known in the art. Such compressor/decompressors convert analog video information into compressed digital information, and vice-versa. The compressed digital information may be stored on hard disk for use at a later time.

A computer implemented control system may include one or more output devices. Examples of output devices include a cathode ray tube (CRT) display, liquid crystal displays (LCD) and other video output devices, printers, communication devices such as a modem or network interface, storage devices such as disk or tape, and audio output devices such as a speaker.

A computer implemented control system also may include one or more input devices. Example input devices include a keyboard, keypad, track ball, mouse, pen and tablet, communication devices such as described above, and data input devices such as audio and video capture devices and sensors. A computer implemented control system is not limited to the particular input or output devices described herein.

It should be appreciated that one or more of any type of computer implemented control system may be used to implement various embodiments described herein. Embodiments may be implemented in software, hardware, firmware, and/or any combination thereof. The computer implemented control system may include specially programmed, special purpose hardware, for example, an application-specific integrated circuit (ASIC). Such special-purpose hardware may be configured to implement one or more of the methods, steps described above as part of the computer implemented control system described above or as an independent component.

A computer implemented control system and components thereof may be programmable using any of a variety of one or more suitable computer programming languages. Such languages may include procedural programming languages, for example, C, Pascal, Fortran and BASIC, object-oriented languages, for example, C++, Java and Eiffel and other languages, such as a scripting language or even assembly language.

Methods may be implemented using any of a variety of suitable programming languages, including procedural programming languages, object-oriented programming languages, other languages, and/or combinations thereof, which may be executed by such a computer system. Such methods can be implemented as separate modules of a computer program, or can be implemented individually as separate computer programs. Such modules and programs can be executed on separate computers.

Such methods, either individually or in combination, may be implemented as a computer program product tangibly embodied as computer-readable signals on a computer-readable medium, for example, a non-volatile recording medium, an integrated circuit memory element, and/or a combination thereof. For each such method, such a computer program product may comprise computer-readable signals tangibly embodied on the computer-readable medium that define instructions, for example, as part of one or more programs, that, as a result of being executed by a computer, instruct the computer to perform the method.

As described herein, a controller may be in electrical communication with one or more sensors and/or may be used to provide feedback to control a property (e.g., a property determined by the one or more sensors) inside and/or around one or more droplets in the devices described herein. The controller may be used, for example, to maintain and/or adjust one or more parameters such as a particular pH, temperature, amount and/or concentration of dissolved oxygen, amount and/or concentration of carbon dioxide, amount or concentration of glucose, amount and/or concentration of a particular protein, and/or amount and/or concentration of particular a metabolite. The controller may also be in electrical communication with one or more components of the digital microfluidic device (e.g., an actuator such as a valve, pump) to control the amount of species added to the droplet and/or digital microfluidic system, e.g., with the assistance of feedback from the one or more sensors.

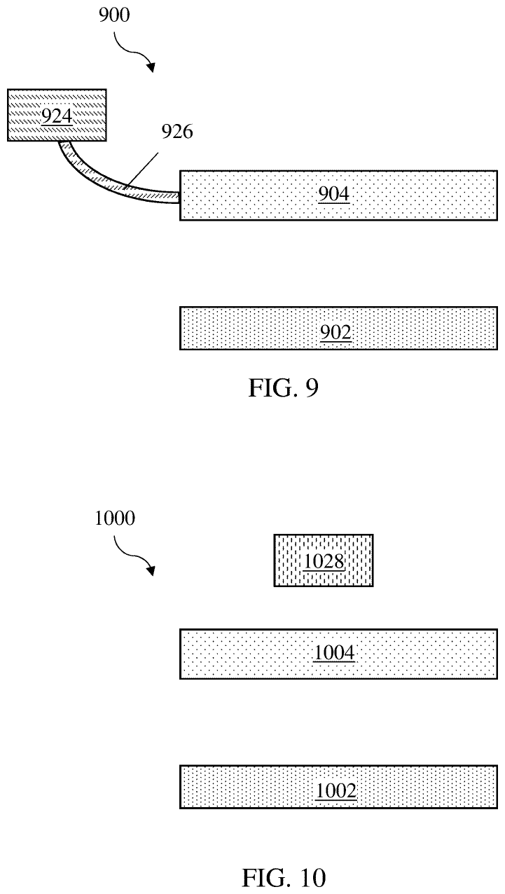

FIG. 9 shows one non-limiting example of a digital microfluidic device in electrical communication with a controller. In FIG. 9, the digital microfluidic device 900 is in electrical communication with a controller 924 via an electrical cable 926.

In some embodiments, a digital microfluidic device comprises an optical detector. It is also possible for a digital microfluidic device to lack an optical detector but be configured to interface, capable of interfacing, and/or interface with an optical detector. An optical detector may be configured to detect one or more optical signals. The interfacing may be achieved via electrical communication, wireless communication, and/or placement of the optical detector such that it is in the path of the light forming the optical signal. A variety of suitable optical detectors may be employed and optical detectors may comprise a variety of suitable components. In some embodiments, an optical detector comprises a microscope (e.g., a fluorescence microscope), a camera, and/or a plate reader. Some optical detectors may be employed to, be capable of, and/or be configured to perform real-time imaging (e.g., of a droplet in a digital microfluidic device). FIG. 10 shows one non-limiting example of a digital microfluidic device 1000 that comprises an optical detector 1028.

Digital microfluidic devices may also comprise, be configured to be in fluidic communication with, be capable of being in fluidic communication with, and/or be in fluidic communication with one or more further components. As one example, in some embodiments, a digital microfluidic device comprises, is configured to be in fluidic communication with, is capable of being in fluidic communication with, and/or is in fluidic communication with a waste receptacle. When present, the waste receptacle may receive waste (e.g., liquid waste), be capable of receiving waste, and/or be configured to receive waste from the digital microfluidic device and/or one or more components thereof.

In some embodiments, one or more components of a digital microfluidic device may be transparent to some wavelengths of light. For instance, a digital microfluidic device may comprise one or more substrates that are transparent (e.g., a base substrate, a top substrate), one or more electrodes that are transparent (e.g., an electrode positioned on a top substrate, an electrode positioned on a base substrate), one or more coatings (e.g., a coating positioned on a top substrate, a coating positioned on a base substrate), and/or one or more dielectrics (e.g., a dielectric positioned on a top substrate, a dielectric positioned on a base substrate). In some embodiments, a base substrate and all components positioned thereon are transparent. It is also possible for a top substrate and all components positioned thereon to be transparent. Advantageously components that are transparent may allow light (e.g., that is generated inside the digital microfluidic device, that the digital microfluidic device is exposed to) to pass therethrough and/or out of the digital microfluidic device. Such light may advantageously be detected by an optical detector positioned outside the digital microfluidic device and/or on a side of the electrode opposite the side on which any droplets are positioned. As an example, with reference to FIG. 10, when the top substrate 1004 is transparent, light generated and/or passing through the space between the top substrate and the base substrate 1002 can also pass through the top substrate and then be detected by the optical detector 1028.

It is also possible for the digital microfluidic devices described herein to comprise one or more components that are opaque to at least some wavelengths of light and/or to lack any components that are transparent to any wavelengths of light. Similarly, it is possible for the digital microfluidic devices described herein to comprise one or more components that are reflective for at least some wavelengths of light. Without wishing to be bound by any particular theory, the inclusion of both one or more reflective components and one or more transparent components may beneficially allow for a relatively large amount of light to be directed to an optical detector. As an example, a digital microfluidic device may comprise a reflective component that is positioned opposite an optical detector and transparent components between the reflective component and the optical detector. Light generated inside such a digital microfluidic device may be reflected and transmitted towards the optical detector.

One example of a digital microfluidic device having a structure comprising both reflective and transparent components is a digital microfluidic device in which a base substrate and any components positioned thereon (e.g., one or more electrodes, a coating, a dielectric) are transparent and the outermost component of a component stack facing the base substrate (e.g., one or more electrodes, a coating, a dielectric, a top substrate) is reflective. Another example of such a digital microfluidic device is a digital microfluidic device in which a top substrate and any components positioned thereon (e.g., one or more electrodes, a coating, a dielectric) are transparent and the outermost component of a component stack facing the top substrate (e.g., one or more electrodes, a coating, a dielectric, a top substrate) is reflective.

In some embodiments, a digital microfluidic device is positioned inside an instrument (and/or capable and/or configured to be positioned inside such an instrument) that can provide an atmosphere suitable for cell culturing. For instance, some digital microfluidic devices may be positioned inside an instrument that can heat and/or cool the digital microfluidic device.

As described above, some embodiments relate to methods, such as methods that digital microfluidic devices are configured to perform, are capable of performing, and/or perform. Further details regarding such methods are provided below.

In some embodiments, a method comprises translating a droplet. The droplet may be translated to, from, and/or across a plurality of locations. The locations that a droplet is translated to, from, and/or across may be locations at which one or more actions are configured to take place, are capable of taking place, and/or take place. It is also possible for such locations to be locations that are particularly suitable for one or more actions to take place. As one example, in some embodiments, a droplet is translated to, from, and/or across a location associated with a sensor. Locations that are associated with a sensor may allow for the sensor to sense one or more properties of the droplet. The association may comprise contact between the sensor and the droplet or may lack such contact. As one example of the latter, a location that is associated with a sensor may be in an optical pathway forming a portion of the sensor and/or from which the receives light (e.g., a location may be in the optical pathway of an IR sensor that detects the temperature of the location). As another example of the latter, a location that is associated with a sensor may be in thermal communication with a sensor.

As described above, sensed properties may be communicated to a controller and/or may be employed as part of a feedback loop to adjust one or more properties of the droplet and/or the digital microfluidic device. Another example of a location that a droplet may be translated to, from, and/or across is a location that is well-suited for cell culture, which may allow for cells to grow in the droplets. A third example of a location that a droplet may be translated to, from, and/or across is a location in fluidic communication with a sensor. Such locations may be well-suited for storing droplets when not in a location associated with the sensor while also allowing for the droplets to be translated to and/or across the sensor when it is desired to sense one or more properties of the droplet.

In some embodiments, a droplet is translated to, from, and/or across a plurality of locations that each differ from one another in one or more ways. It is also possible for a droplet to be translated to, from, and/or across a plurality of locations that are similar in one or more ways. Some embodiments comprise translating a droplet across a plurality of locations that comprise two or more locations that differ from each other in one or more ways and two or more locations that are similar to each other in one or more ways. Additionally, some locations may be both similar to each other in one or more ways and different from each other in one or more ways. One example of such locations is sensors of different type. In some embodiments, a method comprises translating a droplet across two or more locations, each associated with a sensor (some or all of which may be the same as one or more of the other sensors, some or all of which may differ from one or more of the other sensors in one or more ways). In some embodiments, a method comprises translating a droplet across two or more locations associated with sensors that are configured to sense different properties.

Droplet translation may be performed in a variety of suitable manners. In some embodiments, droplet translation is performed with the assistance of one or more electrodes. As an example, a potential difference may be generated between two electrodes. The potential difference may generate an electric field that applies a force to a droplet, causing it to translate. Potential differences may be applied between a variety of suitable combinations of electrodes. In some embodiments, a potential difference is applied between an electrode positioned on the base substrate and the electrode positioned on the top substrate. It is also possible for a potential difference to be applied between two electrodes positioned on a common substrate (e.g., the base substrate) or for three or more electrodes to be held at different potentials from each other.

It is also possible for droplet translation to be performed without the use of electrodes. As an example, a pressure differential may be employed to cause one or more droplets to be translated. The pressure differential may be supplied by a source of vacuum, overpressure, gravity, and/or contact with a moving part.

Droplet translation may be performed continuously, intermittently, or in a manner that both comprises continuous translation and intermittent translation. For instance, in some embodiments, droplet translation comprises one or more periods of time in which the droplet is stationary. Droplets may be translated such that they are constantly in motion until stopped (e.g., for one or more periods of time in which they are stationary). It is also possible for droplets to be mostly stationary but translated over discrete periods of time separated by stationary intervals. Droplets may be translated randomly or in a pre-determined manner. In some embodiments, droplets are translated in a repeating pattern. After completing a circuit of the repeating pattern, the droplets may be stationary for a period of time or may immediately start another circuit. It is also possible for the repeating pattern to comprise one or more period of time in which the droplet is stationary.

Some methods may comprise sensing a property, such as a property of a droplet positioned in a digital microfluidic device and/or a property of the digital microfluidic device. A variety of suitable properties may be sensed, such as pH, dissolved oxygen content, carbon dioxide content, and/or glucose content. The sensing may comprise measuring the property and/or a value thereof. Sensing may be accomplished by use of a sensor. A sensor may sense and/or measure a property of a fluid with which it is associated and/or in contact (e.g., a droplet, an environment internal to the digital microfluidic device). It is also possible for sensors to sense one or more properties of a droplet and/or a digital microfluidic device in a non-contact manner (e.g., by detecting light, heat, or another signal originating in the droplet and/or the digital microfluidic device).

Sensing a property of a droplet may be performed directly on the droplet. In other words, the droplet having the property to be sensed may be translated to a location associated with a sensor and/or a signal arising originating in the droplet having the property to be sensed may be measured. It is also possible for one or more droplet properties are sensed by splitting a subportion of the droplet from the droplet and then sensing the properties of the subportion of the droplet (e.g., by performing a measurement on the subportion of the droplet). The subportion of the droplet may be split from the droplet by, for instance, application of an electric field to the droplet that causes the droplet to split into at least two subportions of the droplet. In such embodiments, one or more subportions of the droplet may be relatively small and one or more subportions of the droplet may be relatively large. The property or properties of the relatively smaller subportion(s) of the droplet may be sensed by one or more sensors and then the relatively smaller subportion(s) of the droplet may be remerged with the other subportion(s) of the droplet or may be discarded. When sensing a property of the subportion of the droplet alters one or more of its properties, in some embodiments, it may be advantageous to discard that subportion of the droplet instead of remerging it with the other subportion(s) of the droplet. This may prevent the introduction into the remerged or relatively larger droplet of deleterious species generated during sensing. In such embodiments, it may be desirable for a relatively small subportion of the droplet to be sensed so that a relatively small fraction of the droplet is discarded after sensing.

In some embodiments, a method comprises communicating a sensed property of a droplet and/or a digital microfluidic device to a controller. As described elsewhere herein, this may be accomplished in a wired and/or in a wireless manner. It is also possible for a method to comprise communicating a sensed property of a droplet and/or a digital microfluidic device to a device external to the digital microfluidic device other than a controller. As an example, in some embodiments, a method comprises communicating a sensed property of a droplet to a computer that is not a controller and/or a display that is not a controller. In some embodiments, a method comprises communicating a sensed property of a droplet and/or a digital microfluidic device directly to an operator. This may be accomplished by, for instance, sound, light, or any other signal that could be observed by an operator.

In some embodiments, a method comprises employing a controller to send a digital microfluidic device instructions. Such instructions may be based on one or more properties of the droplet and/or the digital microfluidic device sensed by the sensor. It is also possible for such instructions to be pre-set and/or pre-programmed, triggered by the elapse of time, and/or triggered by one or more other conditions external to the droplet and/or the digital microfluidic device (e.g., loss of power, temperature in excess of a safe value).

Instructions sent by a controller may instruct a digital microfluidic device to take one or more actions. It is also possible for the instructions to instruct the digital microfluidic device to take no action or to halt one or more actions already taking place (e.g., translation of one or more droplets across a plurality of locations). As an example of an instruction to take an action, the instructions may instruct the digital microfluidic device to continue to take one or more actions that are already in progress (e.g., translation of one or more droplets across a plurality of locations). As another example of an instruction to take an action, in some embodiments, the instructions instruct the digital microfluidic device to modify one or more actions that are already in progress and/or to initiate one or more new actions. For instance, the instructions may instruct the digital microfluidic device to modify the manner in which a droplet is being translated by a digital microfluidic device, such as the speed at which it is translated and/or the locations which the droplet is being translated across. It is also possible for the instructions to instruct the digital microfluidic device to begin translating a droplet across a plurality of locations.

In some embodiments, a controller sends the digital microfluidic device instructions to adjust one or more properties of a droplet. For instance, the controller could send the digital microfluidic device instructions to change the pH, media content, glucose content, and/or nutrient content. Droplet property adjustment may be accomplished in a variety of suitable manners. In some embodiments, one or more properties of a droplet are adjusted by supplying the droplet with a reagent from a reagent source. The reagent source may supply a variety of suitable reagents, such as acids and bases (e.g., to adjust the pH of the droplet), media, glucose, nutrients (e.g., amino acids, nucleotides), cells, and/or cell washing solutions.

A reagent source may supply reagents to a droplet by providing a fluid comprising the reagent. In some embodiments, the fluid is in the form of one or more droplets. It is also possible for the fluid to be supplied in a manner other than one or more droplets but be formed into droplets before entering the digital microfluidic device and/or by the digital microfluidic device. Droplets supplied from a reagent source and/or generated from a fluid supplied by a reagent source may merge with the droplet(s) having one or more properties to be adjusted. Upon exposure to the droplet originating from the reagent source, one or more properties of the droplet having one or more properties to be adjusted may be affected by the reagents in the droplet originating from the reagent source. Supplying reagents in the form of droplets of fluid may advantageously allow for relatively facile control over the amount of the reagents supplied. For instance, reagents may be present in the fluid forming the droplet at a known concentration, and the droplet size may be precisely controlled.

In some embodiments, a reagent source may supply a droplet that is split into one or more smaller droplets before being merged with the droplet(s) having one or more properties to be adjusted. As an example, in some embodiments, a voltage may be applied to a droplet supplied by the reagent source that causes the droplet supplied by the reagent source to eject one or more smaller droplets therefrom. The smaller droplet or droplets may be merged with the droplet(s) having one or more properties to be adjusted. In some embodiments, the droplet from which the smaller droplet or droplets are generated is not merged with the droplet(s) having one or more properties to be adjusted.

It is also possible for a reagent source to supply reagents to a droplet in a manner other than by droplet merger. As one example, in some embodiments, a reagent source may supply one or more reagents to a location in a digital microfluidic device, and a droplet may then be translated across that location. The reagents may be present in the location in a form other than one or more droplets. For instance, in some embodiments, the reagents may be present in the form of a film of liquid disposed on the location. As the droplet is translated across the film of liquid, the reagents therein may become incorporated into the droplet.

When the above-described technique is employed, the formation of a liquid film at a location may be accomplished in a variety of suitable manners. As one example, in some embodiments, the location may be a location that is wet by the fluid supplied form the reagent source (e.g., it may be hydrophilic if the reagent source supplies a hydrophilic fluid). Flowing a fluid (or a droplet thereof) of a fluid supplied by a reagent source across a location wet thereby may result in that location retaining a film of the fluid thereon even if the remainder of the fluid is then flowed away. The remainder of the fluid may be recirculated back to the reagent source, flowed across one or more other locations in the digital microfluidic device, and/or discarded.

Supplying reagents in the form of liquid films has several advantages. One advantage is that the amount of the reagents in the film can be controlled relatively facilely. Flowing a fluid across a location wet by the fluid may result in a film including an amount of the fluid that is relatively reproducible. Another advantage is that reagents can be supplied to droplets in a manner that introduces relatively little additional fluid into the droplet. Some reagents may have a relatively low solubility in a fluid in which they are supplied from a reagent source. Such reagents could be dissolved in the fluid at or close to their solubility limit, and then the fluid could be formed into a film. After these steps, some (or much) of the liquid in the film could be allowed to evaporate from the film before a droplet is flowed across it. This would allow of the reagents present in a fluid supplied from a reagent source to be supplied to the droplet while not also supplying all of the fluid from the reagent source.

In some embodiments, a method comprises performing an assay on a droplet and/or on a subportion of the droplet split from a droplet (e.g., a subportion of the droplet split from the droplet and translated separately from the droplet). Such a subportion of the droplet may be the same subportion of the droplet on which a measurement of a property of the droplet is made or may be a different subportion of the droplet. It is also possible for a subportion of the droplet on which an assay is performed to be the only subportion of the droplet split from a main droplet and/or for only subportions of the droplet on which assays are to be performed to be split from a main droplet. As some assays may change the composition of droplets on which they are performed and/or involve preparation therefor that changes the composition of the droplets on which they are to be performed, performing an assay on a subportion of the droplet may be advantageous for the reasons described elsewhere herein with respect to sensing properties of subportions of the droplet. As one example, in some embodiments, prior to performing an assay on a droplet and/or a subportion of the droplet, any cells present therein are lysed. As another example, in some embodiments, prior to performing the assay on a droplet and/or a subportion of the droplet, any analyte of interest present therein is bound to one or more magnetic beads. It is also possible for any such magnetic beads to be washed after the analyte is bound thereto (e.g., by passing a wash solution, such as a buffer, over the magnetic beads).

In some embodiments, multiple subportions of droplets are split from a common droplet and assays are performed on each subportion. The assays may be the same or different and may be performed at the same time or at different times. As one example, in some embodiments, two or more subportions are split from a common droplet and a different assay is performed on each subportion of the droplet. Methods including the above-described step may be particularly beneficial for determining multiple properties of a droplet at a single point in time. As another example, in some embodiments, a first subportion is split from a droplet at a first point in time on which an assay is performed and then a second subportion is split from the droplet at a second point in time at which the same assay is performed. Methods including the above-described step may be particularly beneficial for determining how a single property of a droplet changes over time. For instance, the results of the assay performed on the first subportion of the droplet may be compared to the results of the assay performed on the second subportion of the droplet.

A variety of suitable assays may be performed on the droplets and subportions of the droplet described herein. Four non-limiting examples of such assays are ELISA assays, enzyme assays, immunoassays, and assays that comprise measuring protein titer (e.g., titer of a recombinant vaccine protein; titer of an antibody, such as a monoclonal antibody and/or IgG). In some embodiments, an assay comprises detecting an antigen via an antibody conjugated to an enzyme. Assays may yield a variety of suitable signals. In some embodiments, an assay is configured to generate, is capable of generating, and/or generates an optical signal. In such embodiments, the optical signal may comprise the absence of light that has been absorbed, may comprise light that has been transmitted (e.g., light of a particular color), and/or may comprise light at a variety of suitable wavelengths (e.g., fluorescent light). It is also possible for an optical signal to comprise spatial information regarding such light. As an example, an optical signal may comprise an image generated from one or more of the above-described types of light. The optical signal may be generated by a species present in the droplet prior to the performance of the assay (e.g., an enzyme produced by cells suspended therein) and/or by its interaction with a species added thereto during the assay (e.g., an enzyme added to the droplet during an assay).

In some embodiments, one or more properties of a droplet is measured by a technique other than an assay or exposure to a sensor. As one example, in some embodiments, a digital microfluidic device may be employed to assess the production of a fluorescent bioproduct in a droplet. This may be accomplished by suspending cells in a droplet that are designed to produce a bioproduct that is conjugated to a fluorescent protein moiety. The production of this bioproduct may be monitored directly by fluorescence.

As described above, some digital microfluidic devices described herein are suitable for generating droplets, manipulating droplets, monitoring droplets, and/or allowing cells to grow in droplets. As also described above, some methods relate to generating droplets, manipulating droplets, monitoring droplets, and/or allowing cells to grow in droplets. Further details regarding droplet properties are provided below.

Some digital microfluidic devices described herein may be suitable for generating, manipulating, monitoring, and/or allowing cells to grow in two or more droplets at a time. It is also possible for two or more droplets to be positioned in a digital microfluidic device at a single point in time. Similarly, some methods may comprise generating, manipulating, monitoring, and/or allowing cells to grow in two or more droplets at a time. In some embodiments, one or more of the methods described herein are performed on two or more droplets during periods of time that overlap. In such embodiments, the droplets may start the method at the same time, proceed through the method at the same rate, and/or terminate the method at the same time. It is also possible for the droplets to start the method at different times, proceed through the method at different rates, and/or terminate the method at different times. When two or more droplets are positioned in a digital microfluidic device and/or a method is performed on two or more droplets, the droplets may be the same as each other or may differ in one or more ways. For instance, the droplets may have different sizes and/or comprise one or more species in different amounts.

The droplets described herein may comprise a variety of suitable components. In some embodiments, a droplet is suitable for performing cell culture. Accordingly, in some embodiments, a droplet described herein comprises cells. Non-limiting examples of cells that may be present in droplets include primary cells, isolated or transformed, cultured cells, eukaryotic cells, prokaryotic cells, animal cells (e.g., blood cells, human leukemia cells, lymphocytes, beta cells, oocytes, egg cells, primary cells, primary bone marrow cells, stem cells, neuronal cells, endothelial cells, epithelial cells, fibroblasts), insect cells, plant cells, bacterial cells, and/or archebacterial cells. Further non-limiting examples of cells that may be present in droplets include Chinese Hamster Ovary cells, NIH-3T3 cells, Hela cells, Jurkat T-cells.

Individual droplets may comprise exclusively one type of cell or may comprise two or more types of cells. When two or more droplets are present in a digital microfluidic device, each droplet may comprise the same type of cell (or same combination of cell types) or two or more droplets may comprise different cells (or different combinations of cell types). Cells present in a droplet may be suspended in therein.

Some droplets may comprise one or more components employed during cell culture. For instance, a droplet may comprise water, media (e.g., basal media, complex media, serum-free media, insect cell media, virus production media), serum (e.g., fetal bovine serum), serum replacements, glucose, nutrients, buffers (e.g., phosphate-buffered saline, HEPES, sodium bicarbonate buffer), salts, surfactants, dyes (e.g., calcein AM, ethidium homodimer-1, fluorescent dyes, fluorogenic dyes, viability dyes), other labeling agents (e.g., quantum dots, nanoparticles), antibiotics, antimycotics, and/or pH indicators (e.g., colorimetric pH indicators, fluorescent pH indicators).

Some droplets may comprise one or more components employed during an assay. For instance, a droplet may comprise drugs, drug lead compounds, toxins, surfactants, transfection reagents, plasmids, supplements, anti-clumping agents, streptavidin, biotin, antibody production enhancers, antibodies, antibody ligands, nucleic acids, nucleic acid binding molecules, enzymes, proteins, viruses, cell process agonists, and/or cell process antagonists.

Some droplets may comprise one or more components that assist with suspending cells therein, such as hydrophilic polymers. Non-limiting examples of suitable hydrophilic polymers include block copolymers, such as block copolymers comprising poly(propylene) oxide and/or poly(ethylene oxide) (e.g., pluronics, such as pluronic F68, pluronic F127).

It is also possible for a droplet to comprise one or more components that assists with its manipulation and/or translation. As one example, in some embodiments, a droplet may comprise a surfactant. The surfactant, and/or at least a portion thereof, may take the form of an external layer on the droplet. Without wishing to be bound by any particular theory, it is believed that the presence of an external layer on a droplet that comprises a surfactant may cause the droplet to have an external surface that is relatively low energy. It is also believed that relatively low energy surfaces may exhibit reduced or minimal spreading on other surfaces, such as surfaces of a digital microfluidic device in which the droplet is positioned. Advantageously, this may allow for droplets to be translated around a digital microfluidic device in a relatively facile manner.

In some embodiments, a droplet and/or a droplet interior is spatially surrounded by a fluid (e.g., in the form of an external layer of the droplet, in the form of a fluid positioned between two substrates enclosing a droplet). In some embodiments, the fluid is air. It is also possible for the fluid to be an oil, such as a hydrophobic oil. Non-limiting examples of suitable hydrophobic oils include silicone oils (e.g., polydimethylsiloxanes, such as DC200; polyphenyl-methylsiloxane, such as AR20), hydrocarbon oils (e.g., tetradecane, hexadecane, octadecane, dodecane, mineral oil, isopar M, vegetable oil), and fluorinated oils (e.g., perfluorohexane (PFH), perfluorinated chemicals (PFCs), perfluorodecalin (PFD), perfluoroperhydrophenanthrene (PFPH), carrier oil supplied by Raindance Technologies, C₉H₅F₁₅O (HFE), fluorinated oils supplied by Novec, CAS No. 86508-42-1 (FC40), N(CF₂CF₂CF₂CF₂CF₃)₃(FC70), CAS No. 86508-42-1 (FC77), C5-C18 perfluoro compounds (e.g., FC3283)).

In some embodiments, a fluid surrounding a droplet does not diffuse into the interior of the droplets that it surrounds. Advantageously, this may facilitate the use of fluids surrounding droplets comprising one or more materials that would not be desirable for inclusion in the interiors of the droplets. As an example, in some embodiments, the interior of a droplet may comprise a cell and a fluid surrounding the droplet may comprise a species toxic to the cell. If the fluid is maintained on the outside of the droplet (e.g., by being immiscible with the droplet interior), then the cell in the interior of the droplet may be unaffected (or minimally affected) by the fluid.

The droplets described herein may have a variety of suitable sizes. In some embodiments, a droplet has a volume of greater than or equal to 0.1 microliter, greater than or equal to 0.2 microliters, greater than or equal to 0.5 microliters, greater than or equal to 0.75 microliters, greater than or equal to 1 microliter, greater than or equal to 1.5 microliters, greater than or equal to 2 microliters, greater than or equal to 3 microliters, greater than or equal to 4 microliters, greater than or equal to 5 microliters, greater than or equal to 6 microliters, or greater than or equal to 8 microliters. In some embodiments, a droplet has a volume of less than or equal to 10 microliters, less than or equal to 8 microliters, less than or equal to 6 microliters, less than or equal to 5 microliters, less than or equal to 4 microliters, less than or equal to 3 microliters, less than or equal to 2 microliters, less than or equal to 1.5 microliters, less than or equal to 1 microliter, less than or equal to 0.75 microliters, less than or equal to 0.5 microliters, or less than or equal to 0.2 microliters. Combinations of the above-referenced ranges are also possible (e.g., greater than or equal to 0.1 microliter and less than or equal to 10 microliters). Other ranges are also possible.

The droplets described herein may comprise a variety of suitable amounts of cells. In some embodiments, a droplet comprises greater than or equal to 10³ cells/mL, greater than or equal to 10⁴ cells/mL, greater than or equal to 10⁵ cells/mL, greater than or equal to 10⁶ cells/mL, or greater than or equal to 10⁷ cells/mL. In some embodiments, a droplet comprises less than or equal to 10⁸ cells/mL, less than or equal to 10⁷ cells/mL, less than or equal to 10⁶ cells/mL, less than or equal to 10⁵ cells/mL, or less than or equal to 10⁴ cells/mL. Combinations of the above-referenced ranges are also possible (e.g., greater than or equal to 10³ cells/mL and less than or equal to 10⁸ cells/mL). Other ranges are also possible.

As described above, in some embodiments, a digital microfluidic comprises a top substrate and/or a base substrate. Further details regarding substrates are provided below.

A variety of suitable substrates may be employed. In some embodiments, one or more substrates present in a digital microfluidic device is or are transparent to at least some wavelengths of light and/or may be opaque to at least some wavelengths of light. Non-limiting examples of transparent substrates include substrates comprising glass (e.g., glass slides) and substrates comprising plastic (e.g., cyclo olefin polymers, polymethyl methacrylate, polycarbonate, polyethylene, polypropylene, and polystyrene).

In some embodiments, a substrate is relatively transparent at one or more wavelengths. For instance, in some embodiments, the substrate and/or coating has a high transparency at wavelengths of greater than or equal to 200 nm, greater than or equal to 250 nm, greater than or equal to 300 nm, greater than or equal to 350 nm, greater than or equal to 400 nm, greater than or equal to 450 nm, greater than or equal to 500 nm, greater than or equal to 550 nm, greater than or equal to 600 nm, greater than or equal to 650 nm, greater than or equal to 700 nm, greater than or equal to 750 nm, greater than or equal to 800 nm, greater than or equal to 850 nm, greater than or equal to 900 nm, or greater than or equal to 950 nm. In some embodiments, the substrate has a high transparency at wavelengths of less than or equal to 1000 nm, less than or equal to 950 nm, less than or equal to 900 nm, less than or equal to 850 nm, less than or equal to 800 nm, less than or equal to 750 nm, less than or equal to 700 nm, less than or equal to 650 nm, less than or equal to 600 nm, less than or equal to 550 nm, less than or equal to 500 nm, less than or equal to 450 nm, less than or equal to 400 nm, less than or equal to 350 nm, less than or equal to 300 nm, or less than or equal to 250 nm. Combinations of the above-referenced ranges are also possible (e.g., greater than or equal to 200 nm and less than or equal to 1000 nm). Other ranges are also possible.

The transmittance of a substrate at wavelengths in one or more of the ranges described in the preceding paragraph may be appreciable. For instance, in some embodiments, a substrate has a transmittance at wavelengths in one or more of the ranges described in the preceding paragraph of greater than or equal to 85%, greater than or equal to 90%, greater than or equal to 95%, greater than or equal to 97.5%, greater than or equal to 99%, greater than or equal to 99.5%, or greater than or equal to 99.9%. In some embodiments, a substrate has a transmittance at wavelengths in one or more of the ranges described in the preceding paragraph of less than or equal to 100%, less than or equal to 99.9%, less than or equal to 99.5%, less than or equal to 99%, less than or equal to 97.5%, less than or equal to 95%, or less than or equal to 90%. Combinations of the above-referenced ranges are also possible (e.g., greater than or equal to 85% and less than or equal to 100%). Other ranges are also possible. The transmittance may be determined in accordance with ASTM D1746-15.

As described above, in some embodiments, a digital microfluidic comprises a top substrate and/or a base substrate. Further details regarding electrodes are provided below.

A variety of suitable electrodes may be employed. In some embodiments, one or more electrodes present in a digital microfluidic device is or are transparent to at least some wavelengths of light and/or may be opaque to at least some wavelengths of light (e.g., it may have a transparency to a wavelength of light in one or more of the ranges described above with respect to substrates and/or a transmittance at such a wavelength in one or more of the ranges described above with respect to substrates). One non-limiting example of a transparent electrode is an electrode comprising indium tin oxide. Non-limiting examples of non-transparent electrodes include electrodes comprising chromium and electrodes comprising gold.

The electrodes described herein may have a variety of suitable sizes. In some embodiments, an electrode (e.g., an electrode positioned on a base substrate) has an area parallel to the substrate on which it is disposed of less than or equal to 1500 mm², less than or equal to 1250 mm², less than or equal to 1000 mm², less than or equal to 750 mm², less than or equal to 500 mm², less than or equal to 200 mm², less than or equal to 100 mm², less than or equal to 75 mm², less than or equal to 50 mm², less than or equal to 20 mm², less than or equal to 10 mm², less than or equal to 7.5 mm², less than or equal to 5 mm², less than or equal to 2 mm², less than or equal to 1 mm², less than or equal to 0.75 mm², less than or equal to 0.5 mm², less than or equal to 0.2 mm², less than or equal to 0.1 mm², less than or equal to 0.075 mm², less than or equal to 0.05 mm², less than or equal to 0.02 mm², less than or equal to 0.01 mm², less than or equal to 0.0075 mm², less than or equal to 0.005 mm², less than or equal to 0.002 mm², less than or equal to 0.001 mm², less than or equal to 0.00075 mm², less than or equal to 0.0005 mm², or less than or equal to 0.0002 mm². In some embodiments, an electrode has an area parallel to the substrate on which it is disposed of greater than or equal to 0.0001 mm², greater than or equal to 0.0002 mm², greater than or equal to 0.0005 mm², greater than or equal to 0.00075 mm², greater than or equal to 0.001 mm², greater than or equal to 0.002 mm², greater than or equal to 0.005 mm², greater than or equal to 0.0075 mm², greater than or equal to 0.01 mm², greater than or equal to 0.02 mm², greater than or equal to 0.05 mm², greater than or equal to 0.075 mm², greater than or equal to 0.1 mm², greater than or equal to 0.2 mm², greater than or equal to 0.5 mm², greater than or equal to 0.75 mm², greater than or equal to 1 mm², greater than or equal to 2 mm², greater than or equal to 5 mm², greater than or equal to 7.5 mm², greater than or equal to 10 mm², greater than or equal to 20 mm², greater than or equal to 50 mm², greater than or equal to 75 mm², greater than or equal to 100 mm², greater than or equal to 200 mm², greater than or equal to 500 mm², greater than or equal to 750 mm², greater than or equal to 1000 mm², or greater than or equal to 1250 mm². Combinations of the above-referenced ranges are also possible (e.g., greater than or equal to 0.0001 mm² and less than or equal to 1500 mm²). Other ranges are also possible.

When a digital microfluidic device comprises two or more electrodes, each electrode may independently have an area parallel to the substrate on which it is disposed in one or more of the ranges provided above.

In some embodiments, an electrode described herein is configured to produce a droplet having a relatively small volume. In some embodiments, an electrode (e.g., an electrode having an area in one or more of the ranges described in the preceding paragraph) is configured to produce a droplet having a volume of less than or equal to 1 mL, less than or equal to 750 microliters, less than or equal to 500 microliters, less than or equal to 200 microliters, less than or equal to 100 microliters, less than or equal to 75 microliters, less than or equal to 50 microliters, less than or equal to 20 microliters, less than or equal to 10 microliters, less than or equal to 7.5 microliters, less than or equal to 5 microliters, less than or equal to 2 microliters, less than or equal to 1 microliter, less than or equal to 750 nanoliters, less than or equal to 500 nanoliters, or less than or equal to 200 nanoliters. In some embodiments, an electrode is configured to produce a droplet having a volume of greater than or equal to 100 nanoliters, greater than or equal to 200 nanoliters, greater than or equal to 500 nanoliters, greater than or equal to 750 nanoliters, greater than or equal to 1 microliter, greater than or equal to 2 microliters, greater than or equal to 5 microliters, greater than or equal to 7.5 microliters, greater than or equal to 10 microliters, greater than or equal to 20 microliters, greater than or equal to 50 microliters, greater than or equal to 75 microliters, greater than or equal to 100 microliters, greater than or equal to 200 microliters, greater than or equal to 500 microliters, or greater than or equal to 750 microliters. Combinations of the above-referenced ranges are also possible (e.g., less than or equal to 1 mL and greater than or equal to 100 nanoliters). Other ranges are also possible.

When a digital microfluidic device comprises two or more electrodes, each electrode may independently be configured to produce a droplet having a volume in one or more of the ranges provided above.

As described above, in some embodiments, a digital microfluidic comprises a coating disposed on a top substrate and/or a base substrate. Further details regarding coatings are provided below.

Coatings disposed on substrates may take a variety of forms. For instance, a coating may take the form of a thin film disposed on a substrate and/or a self-assembled monolayer disposed on a substrate. When present on a substrate, the coating may be covalently bonded to the surface of the substrate or non-covalently bonded to the surface of the substrate. Non-limiting examples of suitable coatings include coatings comprising crystalline, semicrystalline, and/or amorphous fluorinated polymers (e.g., polytetrafluoroethylene, also referred to as Teflon, such as Teflon AF, CYTOP®), fluorinated small molecules, fluorinated oligomers, silanes (e.g., octadecyltrichlorosilane, also referred to as ODTS, and fluorosilanes), and bromo-terminated molecules (e.g., bromo-terminated alkanes).

In some embodiments, a coating disposed on a substrate comprises one or more portions that are hydrophilic. As described above, such portions may be suitable for supporting a liquid film comprising one or more reagents. Portions of a coating that are hydrophilic may comprise one or more extracellular matrix proteins, such as fibronectin, laminin, collagen, and/or elastin. It is also possible for a portion of a coating that is hydrophilic to comprise one or more synthetic polymers, such as poly-L-lysine and/or poly-D-lysine.

In some embodiments, a coating disposed on a substrate comprises one or more portions that are hydrophobic and/or the coating is hydrophobic. The water contact angle of a hydrophobic coating and/or a portion of a coating that is hydrophobic may be greater than or equal to 30°, greater than or equal to 35°, greater than or equal to 40°, greater than or equal to 45°, greater than or equal to 50°, greater than or equal to 55°, greater than or equal to 60°, greater than or equal to 65°, greater than or equal to 70°, greater than or equal to 75°, greater than or equal to 80°, or greater than or equal to 85°. The water contact angle of a hydrophobic coating and/or a portion of a coating that is hydrophobic may be less than or equal to 90°, less than or equal to 85°, less than or equal to 80°, less than or equal to 75°, less than or equal to 70°, less than or equal to 65°, less than or equal to 60°, less than or equal to 55°, less than or equal to 50°, less than or equal to 45°, less than or equal to 40°, or less than or equal to 35°. Combinations of the above-referenced ranges are also possible (e.g., greater than or equal to 30° and less than or equal to) 90°. Other ranges are also possible. The water contact angle may be determined in accordance with ASTM D7334-08 (2013).

In some embodiments, one or more coatings present in a digital microfluidic device is or are transparent to at least some wavelengths of light and/or may be opaque to at least some wavelengths of light (e.g., it may have a transparency to a wavelength of light in one or more of the ranges described above with respect to substrates and/or a transmittance at such a wavelength in one or more of the ranges described above with respect to substrates).

As described above, in some embodiments, a digital microfluidic comprises a dielectric disposed on a top substrate and/or a base substrate. Further details regarding dielectrics are provided below.

Dielectrics protect electrodes from electrolysis, which can damage or remove the patterned electrode material (e.g., Chromium) from the glass. Dielectrics may have a variety of suitable compositions. Non-limiting examples of materials that may be included in the dielectrics described herein include Parylene-C, polyvinylidene difluoride (e.g., PVDF), and silicon oxynitride. Humidity and temperature, though, pose an increased risk of electrolysis to digital microfluidic devices since materials such as Parylene-C degrade in warm, humid conditions. Warm environments in incubator settings may also lead to evaporation on digital microfluidic devices since the small droplet volumes (0.5-10 μL) used can rapidly evaporate in these conditions.

Devices and methods for insulating dielectric layers and for providing supplemental vapor pressure to digital microfluidic devices are described herein. In some embodiments, digital microfluidic devices comprise a sealed enclosure that increases local, relative humidity levels on the droplet interface to reduce the rate of droplet evaporation. This results in stable reagent volumes over the duration of cell culture operations prior to replenishment of the reagents. Also, this embodiment generates regions of high relative humidity for cell culture and low relative humidity to protect electrical contacts and humidity-sensitive features on digital microfluidic devices by mechanically sealing these regions. For example, the regions may be mechanically sealed using gaskets (e.g., rubber gaskets) surrounding the electrical contacts used to supply power to the electrodes of the digital microfluidic devices. In an embodiment, humidity chambers comprising a water bath and/or a salt slurry mixture are used to generate and maintain humidity levels of greater than 80% and greater than 90%.

The digital microfluidic devices described herein may be employed to perform measurements on droplets (e.g., to sense one or more properties of the droplets, to perform one or more assays on the droplets) at a variety of suitable frequencies. In some embodiments, a digital microfluidic device is configured to perform, is capable of performing, and/or performs the measurements at a frequency of at least once per day, at least once every 12 hours, at least once every 6 hours, at least once every 3 hours, at least once every 2 hours, at least once per hour, at least once every 45 minutes, at least once per half hour, at least once every 15 minutes, at least once every 10 minutes, at least once every 5 minutes, at least once every 2 minutes, or at least once every minute. In some embodiments, a digital microfluidic device is configured to perform, is capable of performing, and/or performs the measurements at a frequency of at most once every minute, at most once every 2 minutes, at most once every 5 minutes, at most once every 10 minutes, at most once every 15 minutes, at most once every 45 minutes, at most once every 2 hours, at most once every 3 hours, at most once every 6 hours, or at most once every 12 hours. Combinations of the above-referenced ranges are also possible (e.g., at least once every minute and at most once every 12 hours). Other ranges are also possible.

The digital microfluidic devices described herein may be employed to perform measurements on droplets (e.g., to sense one or more properties of the droplets, to perform one or more assays on the droplets) over a variety of suitable times. In some embodiments, a digital microfluidic device is configured to perform, is capable of performing, and/or performs the measurements over a period of time of at least 1 day, at least 2 days, at least 3 days, at least 5 days, at least 7.5 days, at least 10 days, at least 12.5 days, at least 15 days, at least 20 days, at least 25 days, at least 30 days, at least 31 days, or at least 35 days. In some embodiments, a digital microfluidic device is configured to perform, is capable of performing, and/or performs the measurements over a period of time of at most 40 days, at most 35 days, at most 31 days, at most 30 days, at most 25 days, at most 20 days, at most 15 days, at most 12.5 days, at most 10 days, at most 7.5 days, at most 5 days, at most 3 days, or at most 2 days. Combinations of the above-referenced ranges are also possible (e.g., at least one day and at most 40 days). Other ranges are also possible.

As described above, the digital microfluidic devices described herein may translate droplets, be capable of translating droplets, and/or be configured to translate droplets. The droplets may be translated at a variety of suitable rates. In some embodiments, a droplet is translated at a rate of greater than or equal to 0.2 mm/s, greater than or equal to 0.5 mm/s, greater than or equal to 0.75 mm/s, greater than or equal to 1 mm/s, greater than or equal to 2 mm/s, greater than or equal to 5 mm/s, greater than or equal to 7.5 mm/s, greater than or equal to 10 mm/s, or greater than or equal to 12.5 mm/s. In some embodiments, a droplet is translated at a rate of less than or equal to 15 mm/s, less than or equal to 12.5 mm/s, less than or equal to 10 mm/s, less than or equal to 7.5 mm/s, less than or equal to 5 mm/s, less than or equal to 2 mm/s, less than or equal to 1 mm/s, less than or equal to 0.75 mm/s, or less than or equal to 0.5 mm/s. Combinations of the above-referenced ranges are also possible (e.g., greater than or equal to 0.2 mm/s and less than or equal to 15 mm/s). Other ranges are also possible.

In an alternative embodiment, digital microfluidic devices may be employed to filter cells. Filtration may be used to separate cell media and molecules, such as secreted proteins, organic compounds, or other soluble components from cells. For example, live cells can be filtered using one or more filtration assemblies contained within a digital microfluidic device.

In some embodiments, a digital microfluidic device comprises a filtration assembly having a central component that is liquid permeable, but blocks the translocation of cells or particles through the filtration assembly. The central component is interposed between droplet impermeable segments, such that the droplet(s) present in the digital microfluidic device is restricted from circumventing the filtration assembly through various measures, such as by surrounding the filtration assembly with a physical barrier.

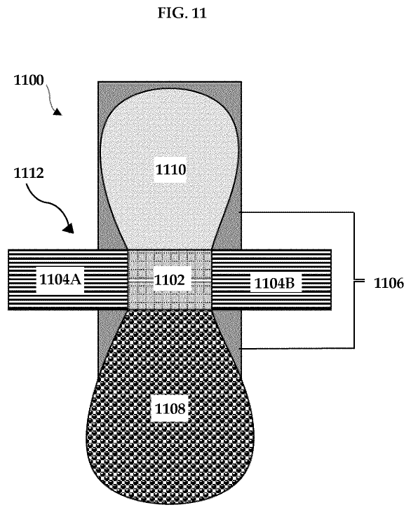

FIG. 11 shows a top view of a non-limiting example of a digital microfluidic device 1100 having a filtration assembly 1112. The filtration assembly 1112 comprises a liquid permeable (and cell/particle impermeable) filter 1102 that is interposed between a pair of liquid impermeable segments 1104A, 1104B. The filter 1102 is positioned over a plurality of electrodes positioned on a side of a base substrate 1106 of the digital microfluidic device 1100. In an embodiment, the filtration assembly 1112 is positioned at the junction of two electrodes. In an alternative embodiment, the filtration assembly 1112 is positioned over a single electrode.

In some embodiments, the filter 1102 comprises paper and each of the impermeable segments 1104A, 1104B comprises paper impregnated with a hydrophobic substance including, but not limited to wax. In this embodiment, a first droplet 1108 contains cells and is pulled through the filter 1102 by actuating an electrode opposite the side on which the first droplet 1108 is positioned and by applying force to a second droplet 1110 located on the opposite side of the filter 1102.

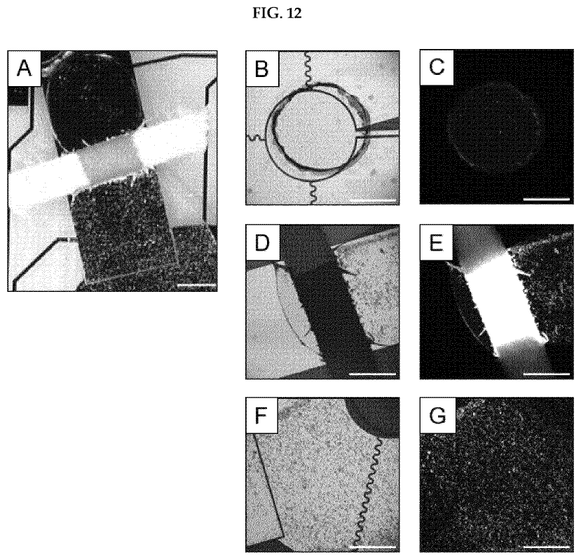

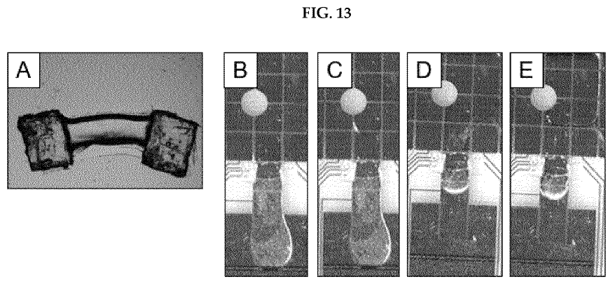

FIG. 12 shows an example of cells filtered on a digital microfluidic device with a paper-based filter. The paper-based filter may be fabricated using Whatman grade 1 or Whatman grade 4 filter paper. The filter paper was patterned with parallel lines (e.g., with 2 mm spacing) of Paraffin wax using a wax pen. The wax patterned paper was then warmed to 100 degrees C. on a hot plate to re-melt the wax such that the wax absorbed through the paper. Cuts were then made orthogonal to the direction of the parallel wax lines creating rectangular filters with wax-impregnated ends.

In the example shown in FIG. 12, the paper-based filter was placed between a top substrate and a base substrate of a digital microfluidic device. To test the paper-based filter, a live cell suspension containing Chinese Hamster Ovary (CHO) cells in cell culture media+0.05% Tetronic 90R4 was pulled through the paper-based filter using electrostatic forces generated by actuating electrodes on the digital microfluidic device. Prior to the test, the CHO cells were stained with Hoescht 338342 fluorescent nuclear stain.

FIG. 12A depicts a Brightfield image of a droplet containing cells being filtered. FIG. 12B depicts a Brightfield image of filtrate captured on a hydrophilic spot on a digital microfluidic top substrate. FIG. 12C depicts a fluorescent image of filtrate captured on a hydrophilic spot on a digital microfluidic top substrate (FL; excitation: 375±14 nm; emission: 460±25 nm). FIGS. 12D and E depict a Brightfield image and a fluorescent image, respectively, of a filter unit after filtering cells. FIGS. 12F and G depict a Brightfield image and a fluorescent image of cell suspension before filtration.

In some embodiments, the material used as a filter is a liquid that is converted to a solid within the digital microfluidic device. The liquid may be positioned using hydrophilic areas patterned on the digital microfluidic top or base substrate. In some embodiments, a digital microfluidic device was prepared for liquid filter deposition by placing two 1.5×1.5 mm squares of 0.2 mm thickness double-sided tape spaced 3 mm apart on either side of an electrode on a base substrate of the device. A top substrate of the digital microfluidic device was prepared with hydrophilic shapes without a hydrophobic coating on the top substrate. This was done by covering the pre-coated top substrate (comprising glass and indium tin oxide) with temperature-resistant masking tape and cutting the desired filter shapes with a laser cutter. The excess tape was then removed to leave masking tape shapes in the desired filter locations.

Next, a hydrophobic coating was then applied such that the cut tape shapes prevented hydrophobic coating in the area designated for filter deposition. For example, the hydrophobic coating was applied by dipping the top substrate with masking tape shapes in a solution of 1% Fluoropel in PFC110 solvent, then baking the coated substrate at 120° C. for 10 min. After cooling, the tape shapes were removed, leaving areas with uncoated indium tin oxide layer in the shape and location where filters were to be deposited. The digital microfluidic top and base substrates were then assembled such that the hydrophilic shapes were juxtaposed with the 1.5×1.5 mm tape squares on the base substrate.

In some embodiments, the filter 1102 comprises a hydrogel and each of the impermeable segments 1104A, 1104B comprises a material including, but not limited to, tape that is coated with a hydrophobic layer. In an embodiment, the gel is translocated and dispensed in liquid form onto an above-mentioned hydrophilic shape on the top substrate, and then cooled to form a solid hydrogel within the digital microfluidic device. In this embodiment, the first droplet 1108 contains cells or particles and is pulled through the filter 1102 by actuating an electrode opposite the side on which the first droplet 1108 is positioned and by applying force to the second droplet 1110 located on the opposite side of the filter 1102. In some examples, the second droplet 1110 may function as a receptacle droplet, which is used to initiate pull-through of the filter 1102 by creating a liquid path through the filter 1102.

FIG. 13 shows an example of cells filtered on a digital microfluidic device with hydrogel-based filters. In this example, a live cell suspension containing Hela cells was filtered on a digital microfluidic device through a gel-based filtration unit. FIG. 13(A) shows a filtration unit stuck to a top substrate on a digital microfluidic device. As shown in (B), a first droplet containing cell suspension was brought to a filter. As shown in (C), a second droplet was brought to the filter from the opposing side. As shown in (D), the first droplet was pulled through the filter using digital microfluidic forces applied to the first droplet on the opposing side. As shown in (E), the first droplet (i.e. filtered droplet) was split from the filter.

In some embodiments, the filter 1102 comprises a porous polymer and each of the impermeable segments 1104A, 1104B comprises a material including, but not limited, tape that is coated with a hydrophobic layer. In an embodiment, the porous polymer was translocated and dispensed in liquid form onto an above-mentioned hydrophilic shape on the top substrate, then polymerized within the digital microfluidic device by exposure to UV light shone through the transparent digital microfluidic top substrate. In some embodiments, digital microfluidic forces are used to pull wash solutions through the polymerized filter to remove unpolymerized monomers and solvents used in the liquid solution. In this embodiment, the first droplet 1108 contains cells or particles and is pulled through the filter 1102 by actuating an electrode opposite the side on which the first droplet 1108 is positioned. In some examples, the second droplet 1110 may function as a receptacle droplet, which is used to initiate pull-through of the filter 1102 by creating a liquid path through the filter 1102. The filter 1102 comprising a porous polymer may be washed to remove cytotoxic elements of the polymer.

In some embodiments, the porous polymer may be amphiphilic since it may be constructed from monomeric compounds having a mixture of hydrophilic and hydrophobic functional groups. For example, the porous polymer disclosed herein may be made using butyl methacrylate as the hydrophobic monomer and glycidyl methacrylate as the hydrophilic monomer. The monomers were mixed in different ratios to adjust hydrophilicity. In other embodiments, the porous polymer may have only hydrophilic groups.

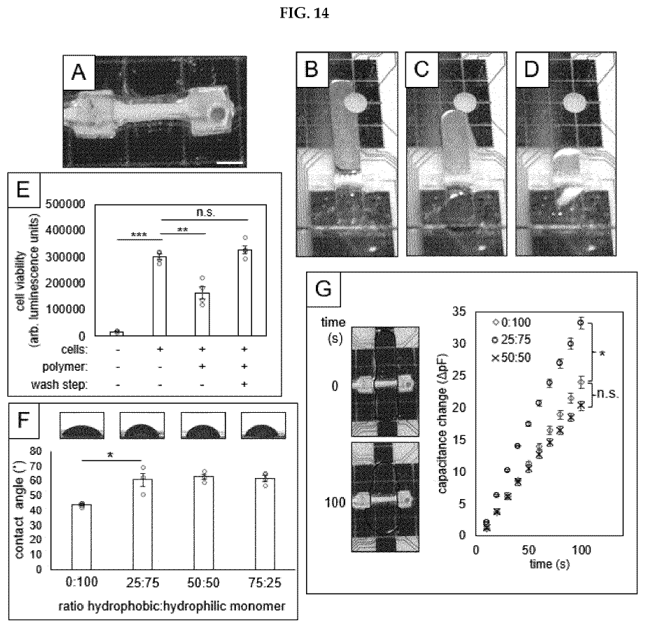

FIG. 14 shows an example of cells filtered on a digital microfluidic device with porous polymer-based filters, and data related to the same. FIG. 14A shows a filtration unit on a digital microfluidic device. In this example, a live cell suspension containing human HeLa cells in media supplemented with 0.05% Tetronic 90R4 was filtered on a digital microfluidic device through a porous polymer-based filtration unit. As shown in FIG. 14B-D, a first droplet was brought to the filter; a second droplet was brought to the filter from the opposing; and cells were concentrated in a smaller droplet after pulling liquid through the filter.

As shown in FIG. 14E, cytotoxicity was tested by incubating Human Hek293T cells with discs of porous polymer with or without prior washing with phosphate buffered saline. In this example, the porous polymer discs were incubated with cells by placing the discs in the upper mesh chamber of a trans-well plate, with cells below. Cell viability was tested with a luminescent viability assay. Porous polymer hydrophilicity was tested by polymerizing a thin layer of porous polymer between two microscope slides pre-coated with the same hydrophobic coating used in digital microfluidic devices (Fluoropel). The two slides were disassembled after polymerization and washed with phosphate buffered saline. A contact angle goniometer was then used to capture and measure the contact angle of water droplets immediately after deposition on the porous polymer layers. Filtration rate was tested by pulling droplets through porous polymer filters on a digital microfluidic device and recording the rate of change of capacitance when six electrodes on the opposite side of the device were actuated.

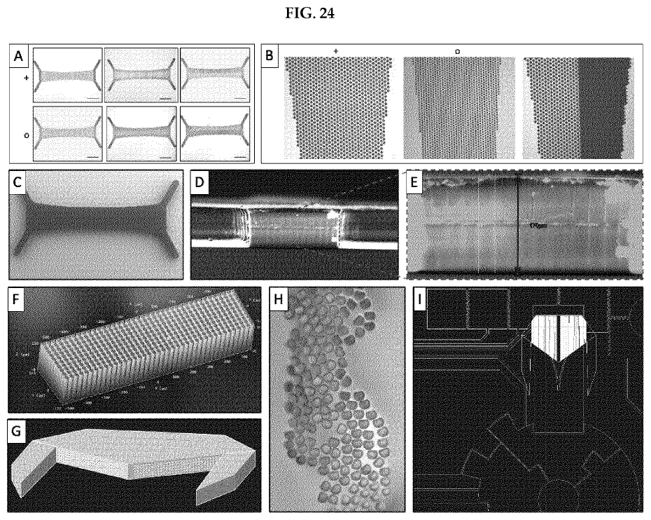

In some embodiments, the filter 1102 comprises a patterned (i.e., microporous) microstructure that is liquid permeable. FIG. 24 shows images of components used for fabrication of patterned microstructure-based filtration assemblies for use on digital microfluidic devices. In an embodiment, patterned microstructure-based filters were fabricated using negative photoresist SU-8 2050 or 2075 on a digital microfluidic top substrate. A chromium-glass photomask was prepared first using a μpg501 mask writer on chromium-glass substrates pre-coated with AZ1500 positive photoresist. After mask writing, the exposed areas of the mask were removed by wet etching with MF312 developer and CEP200 chromium etchant. A 180 micrometer thick layer of SU-8 was deposited on ITO-coated glass substrates by spin coating at 1300 rpm. The SU-8 coated substrate was baked for 65° C. for 7 min, then 95° C. for 45 min. The SU-8 coated substrate was then exposed through the chromium-glass photomask using an OAI mask aligner emitting 350 mJ/cm² UV light for 15 sec. The exposed substrate was baked again at 65° C. for 5 min then 95° C. for 15 min. The substrate was fully immersed in a SU-8 developer for 17 mins to remove the non-exposed SU-8. Finally, the substrate was baked at 65° C. for 7 min, then 95° C. for 20 min, then 180° C. for 10 min.

FIG. 24A shows a fabricated mask with different post shapes (+ and o). FIG. 24B shows a top view of masks for the different posts and a gradient of spacing between the posts. FIG. 24C shows microfabricated posts. FIGS. D and E show cross-section views of 170 μm height posts. FIGS. 24F and G show two different post designs. FIG. 24H shows a top view of microfabricated posts. FIG. 24I shows designs of posts on a digital microfluidic device.

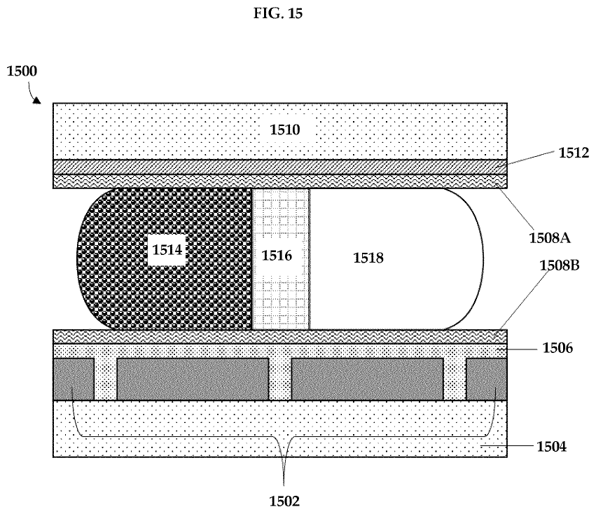

FIG. 15 shows a side view of a non-limiting example of a digital microfluidic device 1500 having a plurality of electrodes 1502. Droplets (e.g., first droplet 1514 and second droplet 1518 in FIG. 15) containing cells are filtered on the digital microfluidic device 1500. The plurality of electrodes 1502 are patterned on a base substrate 1504 and are insulated with a dielectric layer 1506. A top substrate 1510 is spaced apart from the base substrate 1504 so that droplets (e.g., the droplets 1514, 1518 in FIG. 15) may be positioned between them. Such droplets may extend across the full thickness of the space between the bottom and top substrates 1504, 1510 (e.g., like the droplets 1514, 1518 shown in FIG. 15), or may have a height such that they are not in contact with the top substrate (not shown).

In the embodiment shown in FIG. 15, the top substrate 1510 is coated with a conductive layer 1512. Each of the top substrate 1510 and the base substrate 1504 are coated with a hydrophobic layer 1508A and 1508B. As a result, hydrophobic layer 1508A is coated directly to the conductive layer 1512 on the top substrate 1510 and hydrophobic layer 1508B is coated directly to the dielectric layer 1506 on the base substrate 1504. The second droplet 1518 is depicted as being filtered since it has been pulled through a filter 1516 by actuating one or more electrodes 1502 located below the second droplet 1518.

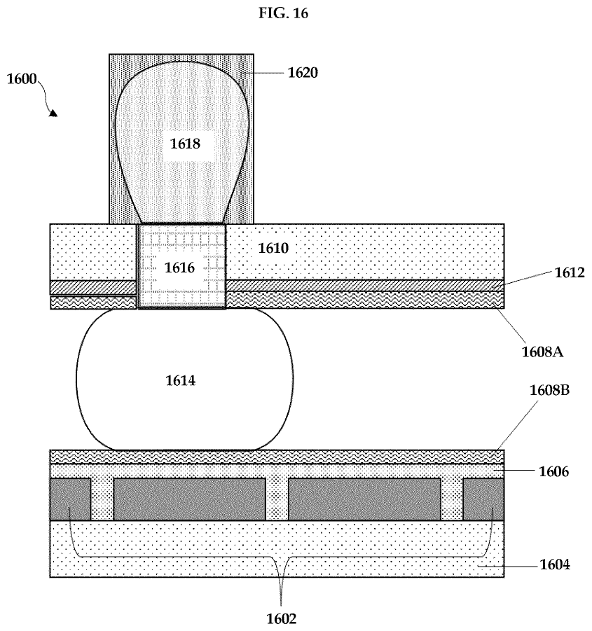

FIG. 16 shows a side view of a non-limiting example of a digital microfluidic device 1600 having a plurality of electrodes 1602 and a vessel 1620. The plurality of electrodes 1602 are patterned on a base substrate 1604 and insulated with a dielectric layer 1606. A top substrate 1610 is spaced apart from the base substrate 1604 so that droplets (e.g., a first droplet 1614 in FIG. 16) may be positioned between them. The top substrate 1610 is coated with a conductive layer 1612. Each of the top substrate 1610 and the base substrate 1604 are coated with a hydrophobic layer 1608A and 1608B. A second droplet 1618 is depicted as being filtered after having been being pulled through a filter 1616 into the vessel 1620 while the first droplet 1614 is positioned between the hydrophobic layers 1608A and 1608B.

In some examples, the vessel 1620 is connected to a device having a suction mechanism. In some examples, a droplet (e.g., the droplet 1614 in FIG. 16) is loaded through the filter 1616 from outside the digital microfluidic device 1600. The first and second droplets 1614, 1618 may each contain cells or particles that are obstructed by the filter 1616.

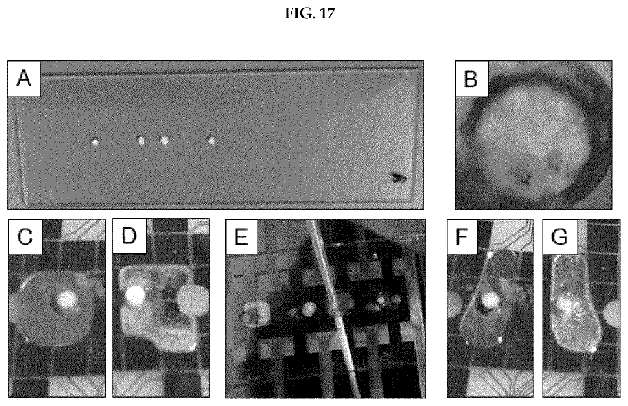

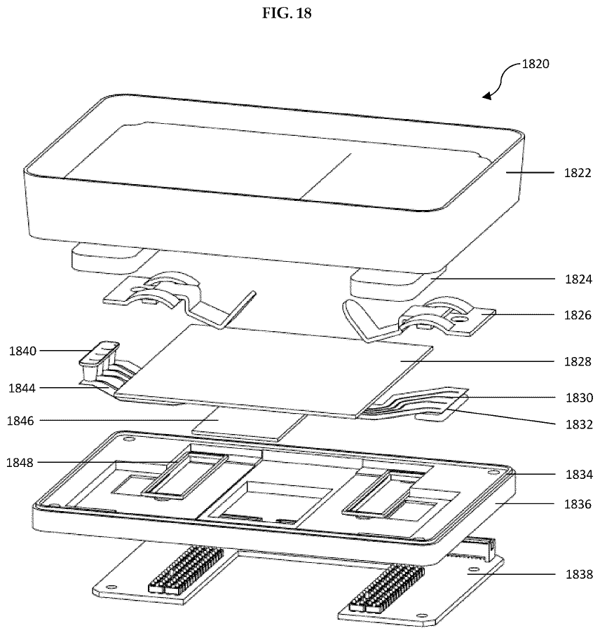

A filtration assembly may be positioned within a hole on either a top substrate or a base substrate of a digital microfluidic device. FIG. 17 shows an example of filtration occurring through holes on a digital microfluidic substrate. In this example, holes were drilled on a digital microfluidic top substrate and filled with porous polymer. Bead suspensions of various diameters were filtered through one or more holes in the digital microfluidic top substrate and beads were resuspended after filtration.

FIG. 17A shows a top substrate of a digital microfluidic device with porous polymer-filled holes. FIG. 17B shows a Brightfield image of a hole in a top substrate of a digital microfluidic device filled with porous polymer. FIGS. 17C and D show 2.8 μm diameter and 30 μm diameter, respectively, bead suspension positioned below a filtration hole in a digital microfluidic device. FIG. 17E shows an image of bead suspension being filtered through a hole with a pipette connected to a vacuum pump. FIGS. 17F and G show 2.8 μm diameter and 30 μm diameter, respectively, beads resuspended after filtration.

In some embodiments and as described further below, a film microchannel may be used to draw out waste from a reservoir (i.e., waste reservoir) using capillary action. In an embodiment, one or more waste chambers located below a digital microfluidic device are used to siphon remaining waste from the digital microfluidic device and into the waste chamber(s). Capillary action is used to prime the film microchannel from the waste reservoir to the waste chamber(s). Hydrostatic pressure from the difference in height between the waste reservoir and the waste chamber(s) drives waste to the waste chamber(s). For example, hydrophilic film channels may be used to facilitate capillary action, thereby providing a surface tension gradient allowing the droplet to move from a hydrophobic surface to a hydrophilic surface.

In some embodiments and as described further below, microchannel film stacks are used to create a seal between base and top substrates, which eliminates a capillary action effect and routes waste into a film microchannel. In embodiments with passive bulk reagent dispensing, an interfacing microfluidic manifold having hydrophobic channels inserted between top and base substrates is present. Driving pressure is achieved passively by means of hydrostatic pressure at a predetermined height of the channel counteracting the pressure drop of the hydrophobic channel. Also, channel restriction at the side of a dispensing electrode acts as a hydrophobic valve to prevent a reagent from escaping into a digital microfluidic device prior to reagent loading on the digital microfluidic device.

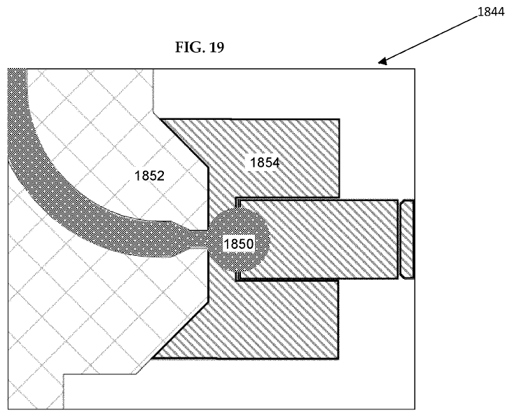

FIG. 18 shows an exploded view of a non-limiting example of a digital microfluidic device enclosure 1820. In operation, the enclosure 1820 may help automate digital microfluidics, maintenance of relative humidity, sealing, passive bulk reagent dispensing, and/or waste removal. The enclosure 1820 comprises pins used to contact pads on a digital microfluidic device via an electrical contact board 1838. For example, silicone gaskets 1834 may seal electrical connectors and/or contact pads of digital microfluidic device interface. The electrical contact board 1838 powers individual electrodes located on a base substrate 1828.

The digital microfluidic device enclosure 1820 also comprises clips 1826, such as spring clips, for securing a digital microfluidic device against the electrical contact board 1838.

The digital microfluidic device enclosure 1820 further comprises a passive bulk reagent dispensing manifold 1844 and a waste removal manifold 1830, which are interposed between the base substrate 1828 and a top substrate counter electrode 1846. A top substrate 1848 is positioned below the top substrate counter electrode 1846. One or more waste removal chambers 1832 attached to the waste removal manifold 1830 can store the waste drawn through the waste removal manifold 1830.

The digital microfluidic device enclosure 1820 further comprises one or more bulk reagent reservoirs 1840 used to store reagent that is used for cell culture and testing. Reagent is passively dispensed onto reservoir electrodes for digital microfluidics operation. The digital microfluidic device enclosure 1820 also comprises a base 1836 and an opposing cover 1822, wherein the cover 1822, encloses the fluidic manifold, humidity chambers 1824, and the digital microfluidic device.

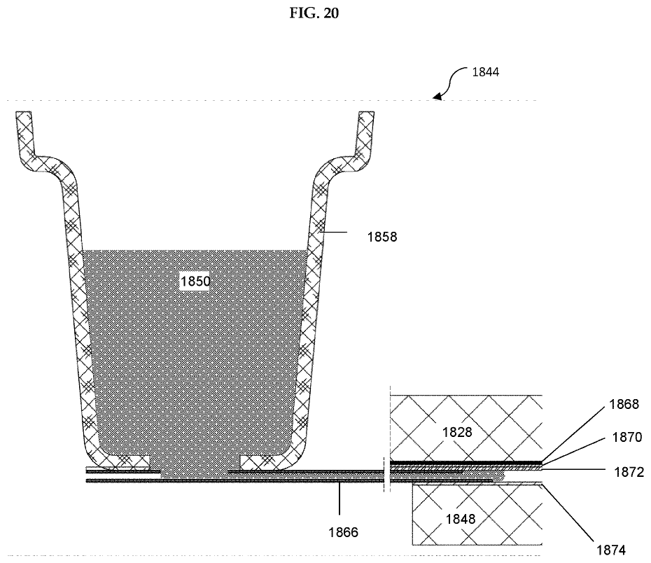

FIGS. 19 and 20 show top and side views, respectively, of the passive bulk reagent dispensing manifold 1844 illustrated in FIG. 18. In this embodiment, reagent 1850 is pushed through a microfluidic channel 1852 and loaded onto a digital microfluidic reservoir electrode 1854. The hydrostatic pressure from one of the bulk reagent reservoirs 1840 provides the driving pressure for the reagent 1850 to overcome the resistance in the microfluidic channel 1852, which is hydrophobic.

The microfluidic channel 1852 is constructed from hydrophobic film material 1866 that is inserted in a gap between the base substrate 1828 and a top substrate 1848. Each of the base substrate 1828 and the top substrate 1848 have a hydrophobic layer 1872, 1874, respectively. A dielectric layer 1870 is positioned between the electrode 1868 and the hydrophobic layer on the base substrate 1828.

The reagent 1850 is contained within a bulk reservoir 1858. The hydrophobic film on the channel 1852 includes adhesive to create a seal between the top and base substrates 1848, 1828 to prevent reagent 1850 from backflowing between the film and either the base substrate 1828 or the top substrate 1848.

In some examples, a restriction at the end of the channel 1852 acts as a valve, whereby the Laplace pressure at the hydrophobic restriction is greater than the hydrostatic pressure. When activated, an electrostatic force from an adjacent electrode 1868 provides a driving force that overcomes the Laplace pressure at the valve, which allows a reservoir electrode 1854 to be filled and manipulated through digital microfluidics operation.

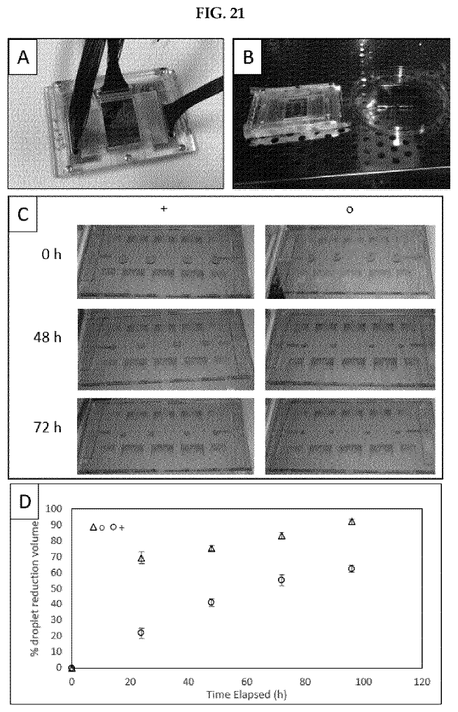

FIG. 21 depicts images of an embodiment of a digital microfluidic device enclosure with a humidity chamber, and data associated therewith. FIG. 21A depicts an enclosure embodiment comprising electronics for electrode actuation. Silicone gaskets are used to seal electrical connectors and contact pads on a digital microfluidic device interface.

FIG. 21B shows an image of a droplet evaporation experiment, wherein the left device is enclosed insider a custom enclosure and the right device is placed inside an open-faced Petri dish. FIG. 21C shows an evaporation experiment of 2 microliter droplets of DMEM over a period of 96 hours inside a custom enclosure (+) and in an open-faced Petri dish (o). FIG. 21D shows a normalized droplet volume reduction over duration of experiment (96 hours). The droplet diameter inside a custom enclosure (+) and inside an open-faced Petri dish was measured every 24 hours over the 96-hour duration (n=4).

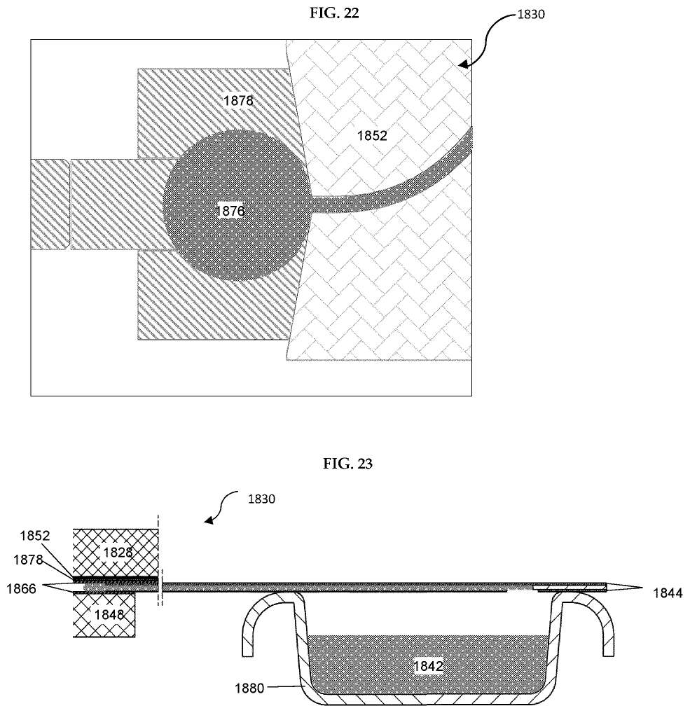

FIGS. 22 and 23 show top and side views, respectively, of the waste removal manifold 1830 of the digital microfluidic device enclosure 1820. In this embodiment, a waste reagent 1876 is drawn from a reservoir electrode 1878 by the microfluidic channel 1852. A hydrophilic microchannel may generate capillary pressure to remove the waste from the reservoir electrode 1878, which primes the microfluidic channel 1852.

The microfluidic channel 1852 is constructed from hydrophobic film material that is inserted in a gap between the base substrate 1828 and the top substrate 1848. The hydrophobic film on the channel 1852 includes adhesive to create a seal between the top and base substrates 1848, 1828 to prevent reagent from flowing between the film and either the base substrate 1828 or the top substrate 1848. The reagent flows to a hydrophilic waste reservoir 1880, which includes waste liquid 1842, and wets the surface. The reagent then flows to the bottom of the waste reservoir 1880 and siphons the reagent in the microfluidic channel 1852 and the reservoir electrode 1878.

The digital microfluidic devices described herein may be employed to perform automated, hands-free assays by implementing the digital microfluidic devices into live cell analysis systems, such as Incucyte®. In an example, a user may program an assay such that a digital microfluidic device can automatically exchange media and deliver reagents at a desired time. Systems, such as Incucyte®, can then conduct automated, real-time imaging and analysis.

In some embodiments, reagent may be stored in a bulk reagent storage reservoir off of the digital microfluidic chip. A fluidic manifold may allow hands-free reagent transfer from the reservoir to respective electrodes. Also, active fluidic actuation can be applied via a piezoelectric pump and passive fluidic actuation may be applied via hydrostatic/capillary pressure.

In some embodiments, an enclosure for the digital microfluidic device may include one or more humidity chambers having water reservoirs. The humidity chambers may minimize the evaporation of droplets to achieve cell growth and high cell viability on the digital microfluidic device in a few droplets.

Conducting automated cell-based assays in systems, such as Incucyte®, provides numerous benefits, including reducing the costs associated with assay and media development, increasing response time, eliminating need for tubes or channels, and improving the accuracy of results. Some of the potential applications include drug screening, LNP screening, potency assays, media development, and reagent screening for iPSC differentiation. While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A digital microfluidic device, comprising:
a base substrate;
a top substrate spaced from the base substrate;
a plurality of electrodes positioned on a side of the base substrate facing the top substrate;
a sensor positioned on a side of the base substrate facing the top substrate;
an electrode positioned on a side of the top substrate facing the base substrate;
a first coating disposed on the plurality of electrodes positioned on the base substrate; and
a second coating disposed on the electrode positioned on the top substrate, wherein:
the electrodes positioned on the base substrate and/or the top substrate are configured to translate a droplet positioned between the base substrate and the top substrate across a plurality of locations;
the plurality of locations comprises a location associated with the sensor;
the sensor is configured to sense a property of a droplet and to communicate the sensed property to a controller;
the digital microfluidic device is in fluidic communication with a reagent source; and
the controller is configured to send the digital microfluidic device instructions to adjust one or more properties of the droplet based on the property sensed by the sensor.

2. A method, comprising:
in a digital microfluidic device comprising a base substrate, a top substrate spaced from the base substrate, a plurality of electrodes positioned on a side of the base substrate facing the top substrate, an electrode positioned on a side of the top substrate facing the base substrate, a first coating disposed on the plurality of electrodes positioned on the base substrate, a second coating disposed on the electrode positioned on the top substrate, and a sensor positioned on a side of the base substrate facing the top substrate:
employing the electrodes positioned on the base substrate and/or the top substrate to translate a droplet positioned between the base substrate and the top substrate across a plurality of locations, wherein the plurality of locations comprises a location associated with the sensor; sensing a property of the droplet with the sensor;
communicating the sensed property of the droplet to a controller;
employing the controller to send the digital microfluidic device instructions to adjust one or more properties of the droplet based on the property sensed by the sensor; and
adjusting one or more properties of the droplet based on the property sensed by the sensor, wherein the one or more properties of the droplet are adjusted by supplying the droplet with a reagent from the reagent source.

3. A digital microfluidic device, comprising:
a base substrate;
a top substrate spaced from the base substrate;
a plurality of electrodes positioned on a side of the base substrate facing the top substrate;
a sensor positioned on a side of the base substrate facing the top substrate;
an electrode positioned on a side of the top substrate facing the base substrate;
a first coating disposed on the plurality of electrodes positioned on the base substrate; and
a second coating disposed on the electrode positioned on the top substrate, wherein:
the electrodes positioned on the base substrate and/or the top substrate are configured to translate a droplet positioned between the base substrate and the top substrate across a plurality of locations;
the plurality of locations comprises a location associated with the sensor;
the sensor is configured to determine the number of cells present in the droplet and/or the viability of cells present in the droplet;
the digital microfluidic device is configured to perform the measurements at a frequency of at least once per hour; and
the digital microfluidic device is configured to perform the measurements over a total period of time of at least 1 day.

4. A method, comprising:
in a digital microfluidic device comprising a base substrate, a top substrate spaced from the base substrate, a plurality of electrodes positioned on a side of the base substrate facing the top substrate, an electrode positioned on a side of the top substrate facing the base substrate, a first coating disposed on the plurality of electrodes positioned on the base substrate, a second coating disposed on the electrode positioned on the top substrate, and a sensor positioned on a side of the base substrate facing the top substrate:
employing the electrodes positioned on the base substrate and/or the top substrate to translate a droplet positioned between the base substrate and the top substrate across a plurality of locations, wherein the plurality of locations comprises a location associated with a sensor configured to determine the number of cells present in the droplet and/or the viability of cells present in the droplet; and
employing the sensor to perform a plurality of measurements of the number of cells present in the droplet and/or the viability of the cells present in the droplet, wherein: the measurements are performed at a frequency of at least once per hour; and the plurality of measurements is performed over a total period of time of at least 1 day.

5. The digital microfluidic device as in claim 1, wherein the droplet comprises cells and/or particles.

6-9. (canceled)

10. The digital microfluidic device as in claim 1, wherein the droplet has a volume of greater than or equal to 0.1 microliter and less than or equal to 10 microliters, and wherein the digital microfluidic device is configured to translate the droplet at a rate of greater than or equal to 0.2 mm/s and less than or equal to 15 mm/s.

11-16. (canceled)

17. The digital microfluidic device as in claim 1, wherein the sensor comprises a pH sensor, a dissolved oxygen sensor, a carbon dioxide sensor, a glucose sensor, a protein titer sensor, a metabolite sensor, an ELISA sensor, and/or a sensor for lactate, glutamine, glutamate, ammonium, and/or potassium.

18-23. (canceled)

24. The method as in claim 2, wherein sensing a property of the droplet comprises performing a measurement on a subdroplet split from the droplet.

25. The method as in claim 24, wherein the subdroplet is translated separately from the droplet.

26. (canceled)

27. The method as in claim 2, wherein sensing the property of the droplet comprises sensing the droplet's pH.

28-33. (canceled)

34. The method as in claim 2, wherein adjusting the property of the droplet comprises adjusting its pH, its media content, its glucose content, and/or its nutrient content.

35-41. (canceled)

42. The method as in claim 2, further comprising performing an assay on the droplet, wherein the assay is an enzyme assay.

43. The method as in claim 2, further comprising performing an assay on a subdroplet split from the droplet.

44. The method as in claim 42, wherein, prior to performing the assay, any cells present in the droplet are lysed.

45-52. (canceled)

53. The method as in claim 42, wherein the assay is configured to generate an optical signal.

54. The method as in claim 53, wherein the optical signal is the absence of light that has been absorbed.

55. The method as in claim 53, wherein the optical signal is fluorescent light.

56. The method as in claim 53, wherein the digital microfluidic device is configured to interface with an optical detector configured to detect the optical signal.

57-58. (canceled)

59. The digital microfluidic device as in claim 1, wherein a dielectric is positioned in between the plurality of electrodes positioned on the base substrate and the first coating.

60. The digital microfluidic device as in claim 1, wherein the electrodes in the plurality of electrodes positioned on the base substrate comprise indium tin oxide.

61-63. (canceled)

64. The digital microfluidic device as in claim 59, wherein the base substrate, electrodes, dielectric and/or first coating are transparent.

65-67. (canceled)

68. The digital microfluidic device as in claim 1, wherein the top substrate, electrode positioned on the top substrate, and/or the second coating are transparent.

69-70. (canceled)

71. The digital microfluidic device as in claim 1, wherein the first coating and/or the second coating comprises Parylene C, silicon oxynitride, and/or poly(vinylidene difluoride).

72-73. (canceled)

74. The digital microfluidic device as in claim 1, wherein the device comprises a filter to filter droplets containing cells within the device.

75-82. (canceled)
